(19) United States
(12) Patent Application Publication
(10) Pub. No.: US 2022/0344019 A1
(43) Pub. Date: Oct. 27, 2022

(54) MEDIA AND MEDICAL DEVICE ECOMMERCE SYSTEM AND METHODS

(71) Applicant: Britta Kay Carlson, Marquette, MI (US)

(72) Inventor: Britta Kay Carlson, Marquette, MI (US)

(21) Appl. No.: 17/726,447

(22) Filed: Apr. 21, 2022

Related U.S. Application Data
(60) Provisional application No. 63/177,525, filed on Apr. 21, 2021.

(51) Int. Cl. G16H 20/00; G06Q 30/06; G06Q 10/10

(52) U.S. Cl. CPC G16H 20/00; G06Q 30/0641; G06Q 30/0633; G06Q 10/1095

(57) ABSTRACT

A medical media and medical device ecommerce system comprising a software platform that operates on computing devices connected through a network. Medical professionals and other qualified individuals, and member of the general public can establish accounts on the system through client computer terminals. Value Media, media having intellectual value to other clinicians or to members of the public is onboarded, IP validated, and stored on the system and available for purchase by other medical professionals and members of the public using an ecommerce module in the system that rewards authors of the uploads. A consortium module provides peer review of the value media by others so qualified. A job board module provides to match and schedule medical professionals and those seeking medical treatment according to various filters for in-home treatment or treatment sessions at cooperative service centers. The system also provides an integrated system for CE testing and credit.

This application claims priority to Provisional Patent Application No. 63/177,525 filed Apr. 21, 2021, the entire disclosure of which is hereby incorporated by reference and relied upon.

### BACKGROUND OF THE INVENTION

### Field of the Invention

The invention relates generally to systems for dissemination of media, and more particularly to dissemination of validated, copyrighted, and professional licensure vetted media and products between healthcare providers through a web-based ecommerce system. The invention also relates to systems for matching and scheduling medical professionals and those seeking medical treatment and systems for CE testing.

### Description of Related Art

Teachers-Pay-Teachers® is an online educational resource that enables teachers to share their expertise and resources. The site provides a means for educators to post curriculum materials they have created for their classes on the Teachers-Pay-Teachers® site for purchase by other teachers who will benefit from this work. This has been a useful tool for some teachers, however, often the materials posted by other educators have undergone little to no vetting leading to questionable materials being posted on the site. For example, teachers are tagging and/or labeling documents as therapy, which were created by a teacher, not a licensed therapist, therefore, disseminating falsely represented medical content. Furthermore, the information does not have the opportunity for validation, continuous improvement, or peer-review for accuracy of medical content. In addition, copyright issues have reportedly been a problem whereby materials created by a third party have undergone slight modifications and reposted on the site for resale. Due to these and other problems, original content creators can easily fall victim to copyright infringement and downloaders may receive false and/or outdated information from non-vetted sources.

What is needed is a web-based system for ecommerce dissemination of media created and vetted by healthcare practitioners to ensure copyright ownership and eliminate redundancy via implementation of an existing coded classification labeling system along with the availability of continuous improvement of content by industry experts. The cost of current systems is dissemination of otherwise potentially invalid and/or unprotected intellectual property of healthcare professionals and belief by in this media by uneducated viewers. Healthcare practitioners such as physical therapists (PTs), occupational therapists (OTs), and speech-language pathologists (SLPs) routinely create materials beneficial to a vast array of tasks related to their work. These clinical tasks can include for example: development of evaluation forms to assess various pathologies, development of clinician treatment programs to assist the patient in overcoming these conditions, development of home exercise programs to regain lost strength and mobility and balance at home, and effective teaching materials to help the patient understand their condition to list just a few. What is needed is a web portal accessible by both clinicians and patients. In addition, what is needed is an ecommerce system to disseminate medical devices that complement home programs a therapist or other clinician may assign to a patient that the clinician has validated and approved. Further needed, is a system for the validation and review of the media for clinical efficacy that are uploaded to the system as well as validation of the medical devices that are prescribed with the therapies. What is also needed, is a system for validating the intellectual property rights related to media uploaded by clinicians.

What is also needed is a concurrent system to match and schedule medical professionals and those seeking medical treatment that has the ability for patients to find independent therapists, but also for therapists to find patients. Finally, what is needed are systems to administer CE testing to medical professionals and to patients, their care givers, and others who wish to build their skills and qualifications. Solutions for these problems and other aspects of the invention are discussed below.

### SUMMARY OF THE INVENTION

Disclosed are embodiments of a media and complementing medical device ecommerce system and methods of use which is referred herein as the ‘web system’.

In one form, a media and complementing medical device ecommerce system is referred to herein as a web system. It comprises a software platform that operates on computing devices connected through a network over the internet (world wide web).

In one form, the web system operates from a server system through a network to serve a plurality of client systems.

In one form, a computing environment comprises components such as a video and touch device, keyboard input device, mouse device, computer processor, computer memory, a network interface, a web browser, and an operating system.

In one form, the web system is controlled by an administrator computing system through a network.

In one form, the web system comprises user facing options on a display of the system that are selectable by a user who is typically a medical professional (also known as a healthcare provider) or a member of the general public. Users who upload value media are sometimes referred to as contributors.

In one form, user facing options include options for establishing an account, logging into the web system, and establishing a clinical segment for onboarding value media. Value media is the media authored by one or more clinicians having intellectual value to other clinicians or patients. The contributor who onboards the value media may also be referred to as a ‘value media contributor’ who is vetted by the platform via their licensure or certification.

In one form, the web system comprises an onboarding module directed to the upload of media to the web system from a contributor source.

In one form of the web system, onboarding comprises one or more of: uploading value media, performing a preliminary validation on the value media, establishing CE credits, and establishing proof of intellectual property ownership.

In one form of the web system, within a consortium module, there may be an additional professional validation of the value media. In addition, there can be validation of the medical devices coinciding with the value media content.

In one form, the web system comprises a consortium module also serves to provide an optional enhanced review of the value media whereby members of a consortium contribute to or make improvements to the value media.

In one form of the web system, following enhanced review, ownership of the intellectual property is revised if necessary to reflect contributions of consortium members.

In one form of the web system, a commission paid to contributors is revised to reflect commissions to be paid to contributors and consortium members who contributed to the value media. This provides a virtual product improvement process, thus increases the value and price of the content while dividing revenue shares of that media among the initial value media contributors and the successive contributors.

In one form of the web system, the consortium assigns diagnostic treatment codes via the International Classification of Diseases, Tenth Revision, (ICD-10) and future revisions to be amended as deemed appropriate by the web system operators and owner.

In one form of the web system, statistical data related to the value media and medical devices is gathered.

In one form of the web system, value media is released for posting to a professional healthcare database for purchase by clinicians and the general public when applicable, i.e.: educational content.

In one form of the web system, value media is posted to an in-home care database for purchase by consumers.

In one form of the web system, statistical data is available for purchase at a statistical data report sales window and downloaded for use.

In one form of the web system, using purchaser facing options, a purchaser can shop online for value media and related medical devices at a shopping window.

In one form of the web system, using purchaser facing options, a purchaser establishes a purchase account through the back end of the web system.

In one form of the web system, using purchaser facing options, a purchaser searches a database of value media and associated medical devices and makes selections of the value media and/or associated medical devices for purchase.

In one form of the web system, using purchaser facing options, a purchaser chooses skins that can be applied to further customize the value media to consumer preferences (such as application of icons/content that relates to their preferred hobbies, activities, holidays, or seasons, etc.). These skins can be modified multiple times for a nominal portion of the initial download with each change of skin applied to the initial content purchased.

In one form of the web system, using purchaser facing options, a purchaser chooses medical devices linked to the value media.

In one form of the web system, using purchaser facing options, payment for the value media and medical devices is processed.

In one form of the web system, the purchased value media can be downloaded.

In one form of the web system, commission to contributors and contributing members of the consortium are processed.

In one form of the web system, the medical device order with payment is sent to the supplier of the device where the device order is fulfilled.

In one form, from a landing page of the web system, a user can establish a professional account or a general public account.

In one form, the general public account user can access home therapy programs posted by a member having a professional account.

In one form, the web system comprises a module for reviewing value media for appropriate content.

In one form, the web system provides for the posting of value media to a professional therapy database or an in-home therapy database.

In one form, the web system comprises a job board module.

In one form, the job board module comprises a therapy matcher and scheduler for in home and service center patients.

In one form, the job board module provides a database of consumers seeking medical treatment that is searchable by a healthcare provider seeking to provide treatment.

In one form, the job board module provides a database of therapists seeking to provide medical treatment that is searchable by a consumer seeking medical treatment.

In one form, the job board module provides an avenue for healthcare providers to market themselves to consumers seeking the services of a healthcare provider.

In one form, the job board module utilizes a maps plugin to isolate potential matches between a healthcare provider and a consumer seeking medical treatment within a specified geographic location.

In one form, the job board module includes scheduling to treatment sessions at therapy service centers.

In one form, the web system comprises an ecommerce module for the sale of value media and processing sales.

In one form, the web system provides CE testing and administering of CE credit.

### BRIEF DESCRIPTION OF THE SEVERAL VIEWS OF THE DRAWINGS

These and other features and advantages of the present invention will become more readily appreciated when considered in connection with the following detailed description and appended drawings, wherein each drawing is according to one or more embodiments shown and described herein, and wherein:

FIG. 1 depicts a block diagram of a process of operation of a media and medical device ecommerce web portal system with focus on onboarding of media;

FIG. 2 depicts a block diagram of a process of operation of a media and medical device ecommerce web portal system with focus on reviewing of content and a consortium module;

FIG. 3 depicts a block diagram of a process of operation of a media and medical device ecommerce web portal system with focus on a job board module;

FIG. 4 depicts a block diagram of a process of operation of a media and medical device ecommerce web portal system with focus on an ecommerce module;

FIG. 5 depicts a block diagram of a process of operation of a media and medical device ecommerce web portal system with focus on a CE testing and credit module;

FIG. 6 depicts a block diagram illustrating various data that can be displayed in a medical professional dashboard module of a media and medical device ecommerce web portal system;

FIG. 7 depicts a block diagram illustrating various data that can be displayed in a general public dashboard module of a media and medical device ecommerce web portal system;

FIG. 8 depicts a typical computing environment utilized to support operation of a media and medical device ecommerce web portal system;

FIG. 9 depicts various computing devices that can be utilized and communicate over a network to support operation of a media and medical device ecommerce web portal system;

FIG. 10 depicts a wireframe of a landing page that can be utilized in a media and medical device ecommerce web portal system;

FIG. 11 depicts a wireframe of a log in page that can be utilized in a media and medical device ecommerce web portal system;

FIG. 12 depicts a wireframe of a webpage for selecting preferences in a media and medical device ecommerce web portal system;

FIG. 13 depicts a wireframe of a webpage showing various subscription plans available to an account user in a media and medical device ecommerce web portal system;

FIG. 14 depicts a wireframe of an ecommerce cart webpage in a media and medical device ecommerce web portal system;

FIG. 15 depicts a wireframe of a payment selection window webpage in a media and medical device ecommerce web portal system;

FIG. 16 depicts a wireframe of a webpage showing value media available for purchase in a media and medical device ecommerce web portal system;

FIG. 17 depicts a wireframe of a webpage showing value media available for purchase with reviews in a media and medical device ecommerce web portal system;

FIG. 18 depicts a wireframe of a webpage showing a review page for reviewing value media in a media and medical device ecommerce web portal system;

FIG. 19 depicts a wireframe of a webpage showing user information related to a user's profile;

FIG. 20 depicts a wireframe of a webpage used to change a user's password;

FIG. 21 depicts a wireframe of a webpage showing CE certificates that a user has earned in a media and medical device ecommerce web portal system;

FIG. 22 depicts a wireframe of a webpage showing documents available for peer review in a media and medical device ecommerce web portal system;

FIG. 23 depicts a wireframe of a webpage showing various medical treatment opportunities available to medical professionals in a media and medical device ecommerce web portal system;

FIG. 24 depicts a wireframe of a webpage of medical treatments sought by consumers at a neutral site in a media and medical device ecommerce web portal system;

FIG. 25 depicts a wireframe of a webpage showing details of a medical need in a media and medical device ecommerce web portal system;

FIG. 26 depicts a wireframe of a webpage showing at the bottom statistical data related to onboarded documents in a media and medical device ecommerce web portal system;

FIG. 27 depicts a wireframe of a webpage showing user's various financial accounts for use in ecommerce in a media and medical device ecommerce web portal system;

FIG. 28 depicts a wireframe of a webpage showing management of uploaded documents in a media and medical device ecommerce web portal system;

FIG. 29 depicts a wireframe of a webpage for uploading documents in a media and medical device ecommerce web portal system;

FIG. 30 depicts a wireframe of a webpage for creation of a certification test in a media and medical device ecommerce web portal system;

FIG. 31 depicts a wireframe of a webpage showing successful submission of value media in a media and medical device ecommerce web portal system;

FIG. 32 depicts a wireframe of a webpage for editing of a certification test in a media and medical device ecommerce web portal system;

FIG. 33 depicts a wireframe of a webpage for editing a value media document in a media and medical device ecommerce web portal system;

FIG. 34 depicts a wireframe of a webpage showing value media documents that have been purchased by a user in a media and medical device ecommerce web portal system;

FIG. 35 depicts a wireframe of a webpage showing a first test page of a CE test in a media and medical device ecommerce web portal system;

FIG. 36 depicts a wireframe of a webpage showing a CE test in a media and medical device ecommerce web portal system;

FIG. 37 depicts a wireframe of a webpage showing passage of a CE exam in a media and medical device ecommerce web portal system.

### DETAILED DESCRIPTION OF SELECTED EMBODIMENTS OF THE INVENTION

Select embodiments of the invention will now be described with reference to the Figures. Like numerals indicate like or corresponding elements throughout the several views. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

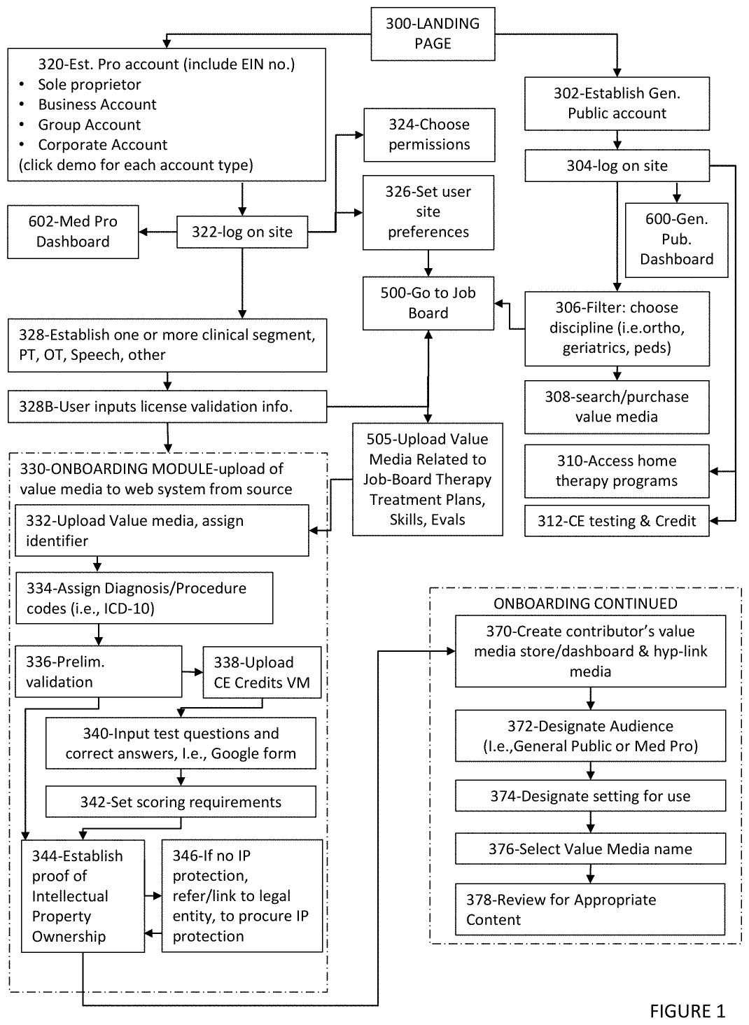

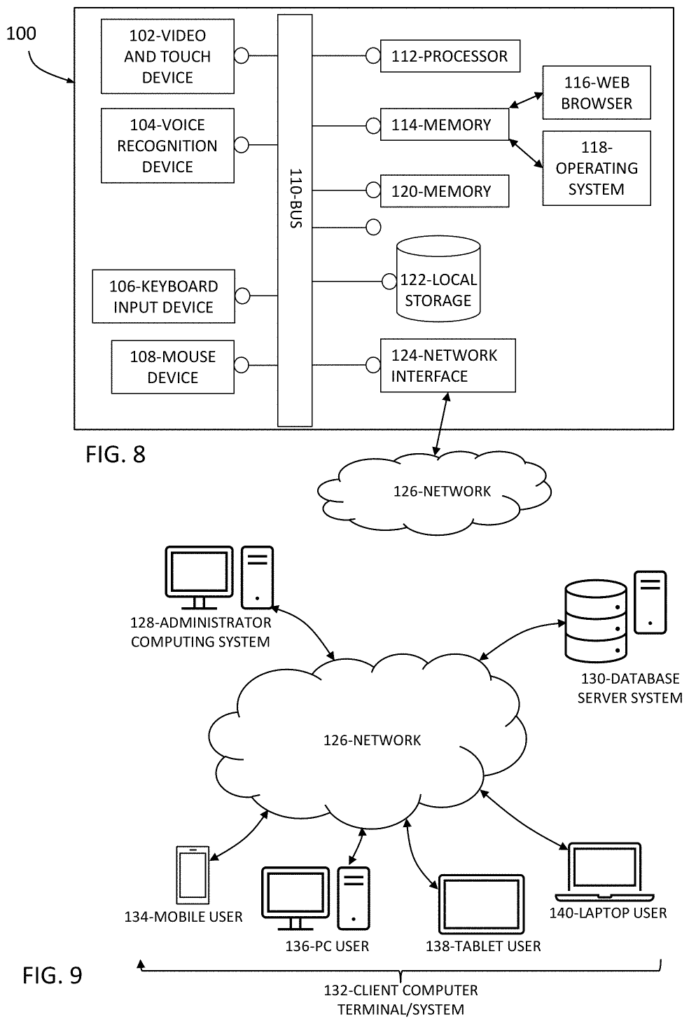

FIGS. 1-7 are block diagram depictions of portions of a preferred embodiment of a process of operation of a media and complementing medical device ecommerce web portal system. This system is referred to as the ‘web system’ herein and is accessed at a web address specified by the owner of the system that directs a user to a landing page 300 of the web system. The web system typically operates on one or more database server systems 130 and can be accessed via any number of client system computing devices 132 through a network such as the internet (FIGS. 8 and 9). The web system can be configured to serve any one or more of: the general public, medical establishments, medical professionals, and patients of those medical professionals. In one form, the web system is configured as a marketplace of medical related information such as that used by physical therapists (PT), occupational therapists (OT), and speech therapists (SLP).

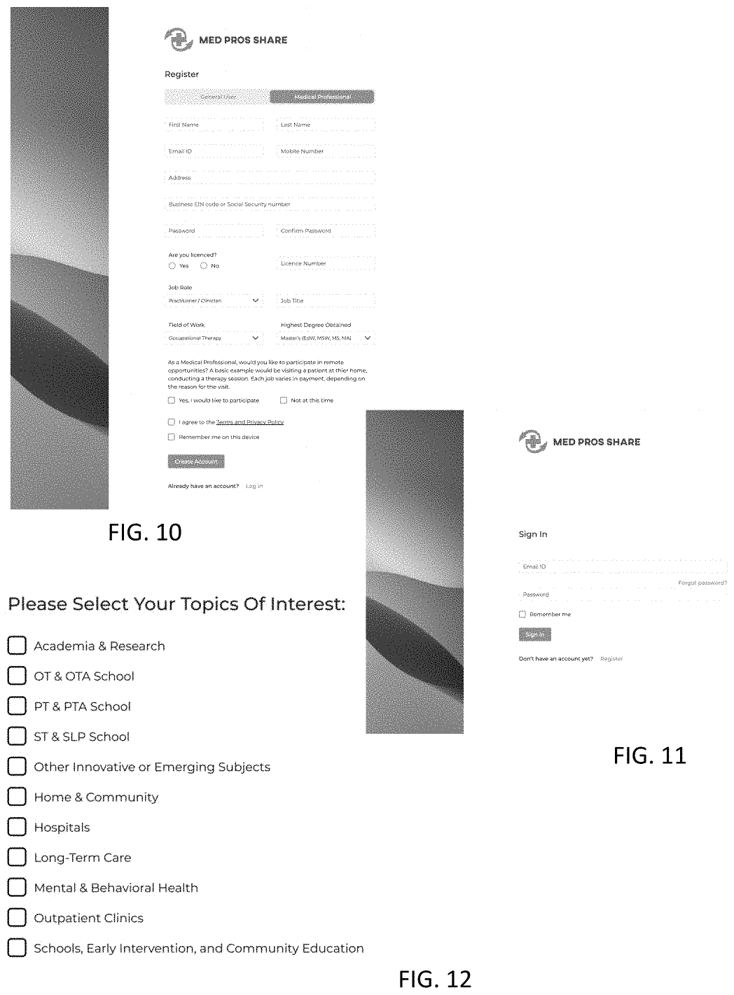

Value Media (VM) is the media authored by one or more clinicians, medical professionals, or other qualified person(s) having intellectual value to other clinicians or patients. Users of the web system are termed ‘users’ herein. Users who upload value media are sometimes referred to as a ‘contributor’. A contributor or ‘value media contributor’ is typically a clinician although they could be a representative of a business entity. Users of the web system utilize a client computer terminal 132 having a display that provides the user access to the world-wide web. Upon arriving at a landing page of the web system, the user is offered a first set of user facing options for establishing an account 300 on the web system. An example wireframe of one landing page is illustrated in FIG. 10 to register as a ‘general user’ (an individual in the general public) or as a ‘medical professional’. This first set of user facing options can also include an option to establish a professional account 320 (FIG. 1) as a ‘sole proprietor’, a ‘business account’, a ‘group account’, or a ‘corporate account’. A ‘sole proprietor’ can be for example, a single clinician, whereas a ‘business account’ can refer to private practice, for example, encompassing several therapists associated with a business who all may be contributors of value media owned by the ‘business’. A ‘group’ account refers to several sole proprietors that cooperate as a group but not as a business. A ‘corporate’ account refers to a corporation that owns two or more clinics with potential value media contributors at each clinic. Additional user facing options can include on screen demos showing how each account type works. It is preferred that during the step of establishing an account, an EIN or social security number corresponding to the individual or group is obtained and saved to a memory device in the web system for later use when necessary for tax purposes.

Once the account is established, the user is faced with a new contributor facing option to log on to the site 302. A wireframe example of a log in page is depicted in FIG. 11. This may include for example a field for entering a username unique to the clinician and a field to enter a password. Once logged in, the user is presented with several additional user facing options. One option includes choosing permissions 324 whereby a client administrator can assign permissions established for members having access through business, group, and corporate accounts. For example, in a business account, some clinicians in the business can be assigned permissions that permit them to upload a variety of media for PT, OT, and SLP, whereas other clinicians can only upload to a single discipline. Furthermore, access for certain groups or corporations can be manipulated by the administrator of the web system to either keep accessibility open to their media or lock certain content which then can only be viewed within the group or corporation's organization.

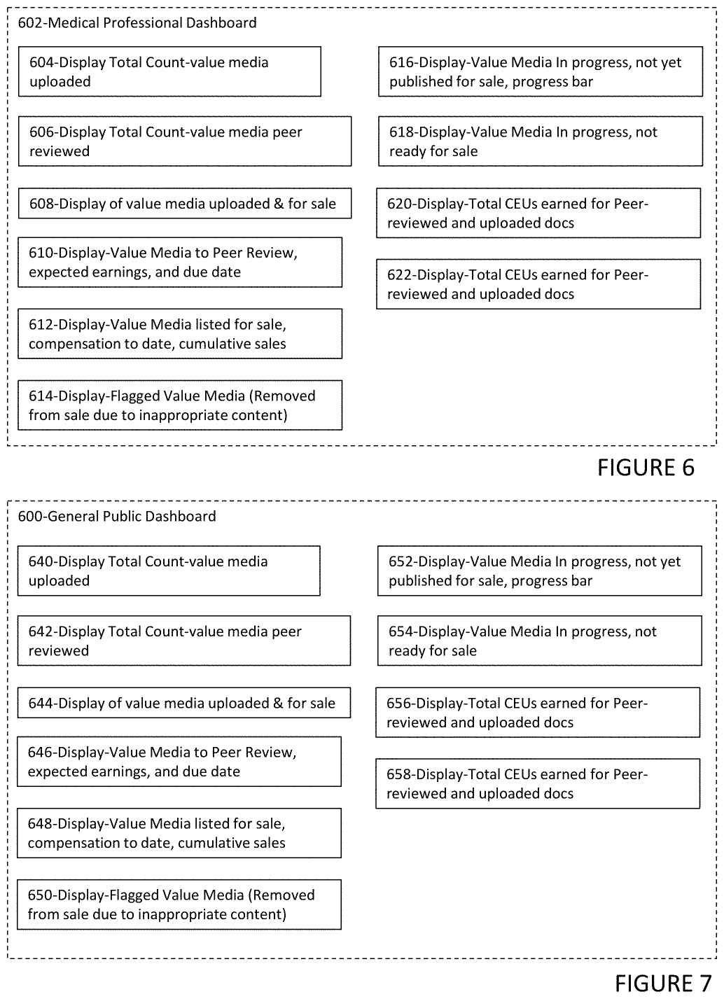

Another user facing option after establishing a professional account on the site is to go to a corresponding medical professional dashboard 602 to view on the user's display various metrics and other information related to their account as depicted in FIG. 6.

Yet another user facing option after log in is for setting user site preferences 326. It is here the user can ‘teach’ the system about preferred displays. For example, if the logged in user is an occupational therapist, activation of preferred preferences in the web system will cause display of material that favors value media of high value to OTs. Likewise, if the user has a special interest in ‘mental and behavioral health’, the web system will favor display of value media related to this topic. FIG. 12 depicts an example wireframe embodiment for selecting site preferences.

Another user facing option following logging on the site is to enter a job board 500 module either directly or after establishing a clinical segment and/or inputting license validation information. The job board 500 will be explained in greater detail in the pages ahead. In addition, yet another user facing option displayed on the user's terminal is to continue on toward onboarding 330 to upload value media. When moving towards onboarding, the contributor faces options to choose the applicable clinical segment for the media(s) to be uploaded 328. For example, the media can be relevant to the clinic practice of PTs, OTs, SLPs, or other clinical disciplines relevant to the care of patients. In some cases, the media for upload is relevant to more than a single discipline, such as for example the ‘Clinical Care of Patient's battling Covid19’. This subject would be relevant to multiple clinical disciplines and therefore can be uploaded and accessed for purchase by clinicians/a business account/group account/or corporate account (purchasers) across a variety of clinical disciplines and/or settings with the option to purchase multiple licenses for distributional use of that media. In step 328B, the user inputs validation of applicable healthcare licensure(s) and/or certifications via, but not limited to, a photo upload and/or inputting a license number along with the state and country in which the license or certification was obtained. In addition, a user can have the option to select interests associated with various medical disciplines or medical conditions such as illustrated in the user facing screen of FIG. 12.

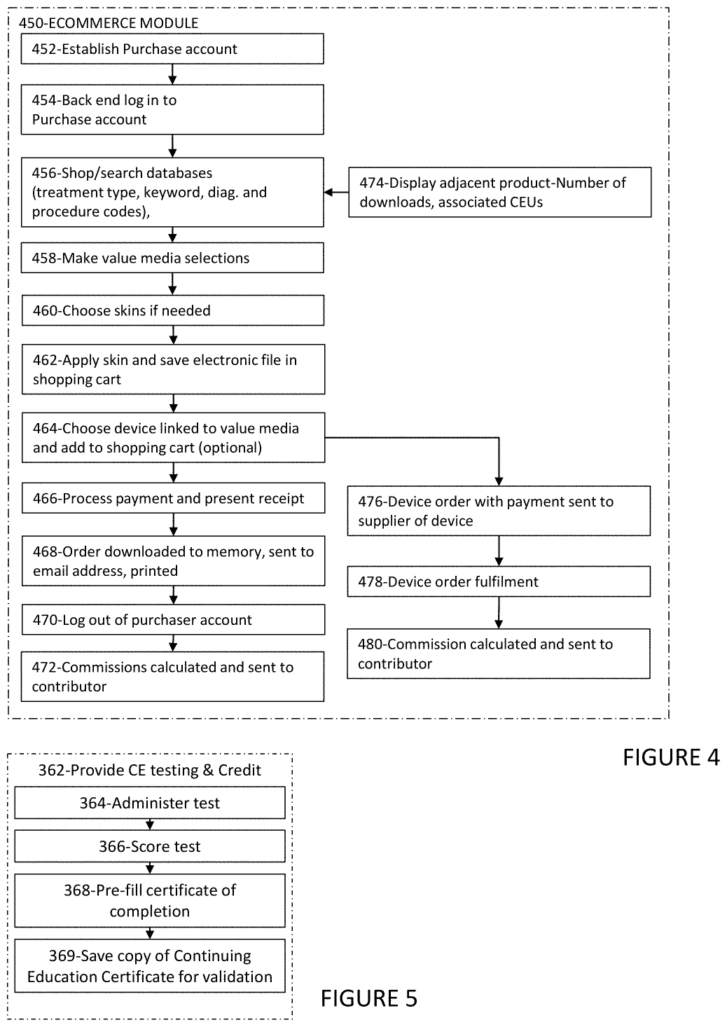

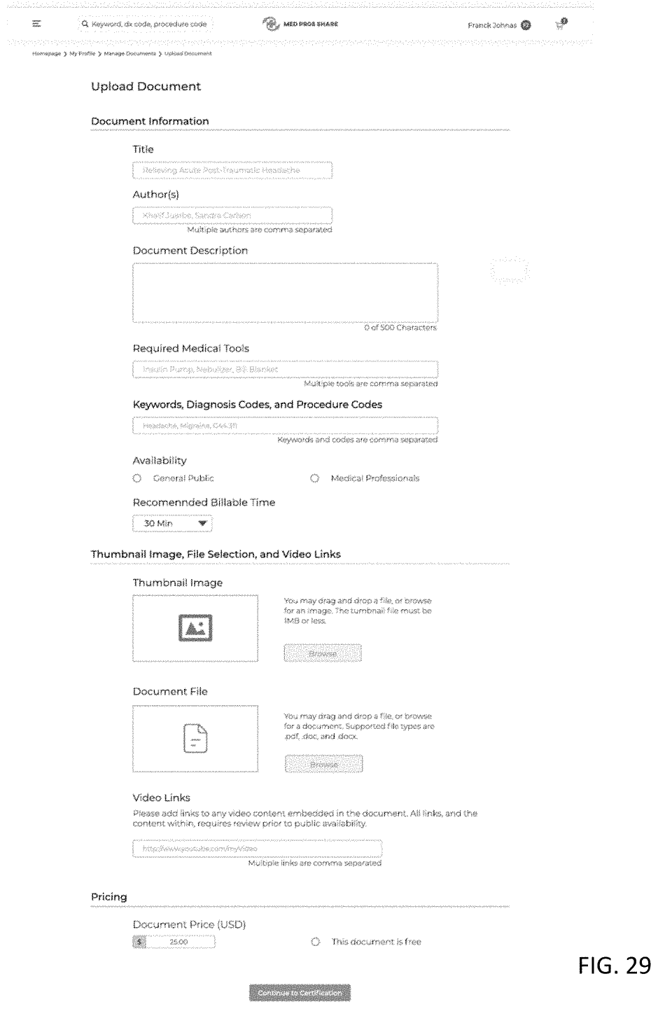

The clinician is then faced with a next set of user (contributor) facing options related to ‘onboarding’ 330 the media which refers to uploading the value media 332 through a client computer terminal 132 through a network 126 to memory devices 120 in the cloud or on servers associated with the web system. Typically, the upload will comprise four parts or steps. The first part is uploading the value media 332. Again, value media is the media authored by a clinician, medical professional, or other qualified person having intellectual value to other clinicians or patients/consumers who are the ‘purchasers’ of the content in an ecommerce module 450 (FIG. 4) of the web system. FIG. 29 is an example wireframe of an upload document page that can be used in the web system. The value media is assigned a numeric code identifier to uniquely identify each uploaded media content. Although not limited to these areas, value media can include for example: specialized evaluation forms for clinical evaluation of patients encountering a specific pathology, specialized treatment protocols for treating these patients, home exercise programs targeting rehabilitation of patients again enduring a specific pathology, and educational materials directed to educating the patient or clinician about various aspects of their care and rehabilitating their condition. Such education materials can include professional continuing education programs that may or may not qualify for continuing education credit. The value media can be in a variety of forms or combination of forms including photographs, text documents, Power Point presentations, photos, illustrations, and any other known in the art means of communication. In addition, in preferred embodiments, the value media is in a modifiable format. For example, a .docx document is modifiable to .pdf, or an Adobe Photoshop file is modifiable to a JPEG format.

In preferred embodiments, the web system includes operations for converting modifiable electronic files into common shareable file types. Note this upload value media step 332 can include user facing options allowing the user to input keywords, designate/limit the value media to an intended audience, add thumbnail images, provide links to the value media such as on YouTube®, add value media pricing, and perform various other options.

The second part of onboarding comprises preliminary assignment of disease codes 334 such as ICD-10 (International Classification of Diseases 10^{th} revision) codes and/or the most recent updated and applicable version of the International Classification of Diseases coding system to the value media. The ICD-10 codes are a standard coding system for use by healthcare professionals, health care organizations, and insurance programs for reporting, documenting morbidity statistics, and billing. These codes are often necessary when a patient and/or clinician pursues reimbursement for clinical work or devices provided to a patient. Assigning this coding, will assist clinicians in finding value media for purchase targeting specific diagnoses and/or treatments. In addition, this information can also be used to calculate effectiveness of various treatments. In some embodiments, step 334 is integrated into the uploading of value media 332 step.

In the third part of onboarding, preliminary validation 336 of the value media is addressed. Typically, this is in the form of a document that provides a history, background, development, and test of the information in the value media uploaded in the first part. For example, value media preliminary validation 336 directed to a specific physical therapy treatment protocol might specify the patient population on which the treatment protocol was utilized, parameters for patient's condition such as pre and post muscular weakness patterns, neurological symptoms, etc. Further, the preliminary validation 336 may include data related to: gender, the age of the patients, frequency and length of treatment, collateral diagnoses, and overall cost from initial evaluation to discharge. In short, the preliminary validation includes information that will assist other clinicians to determine if the respective value media is appropriate for use to their patient population and whether the value media is likely to produce the result intended by the patient and therapist or other clinician. The preliminary validation can also be utilized by the consortium for professional validation of the therapy described in the value media. This will be described in more detail later in this document.

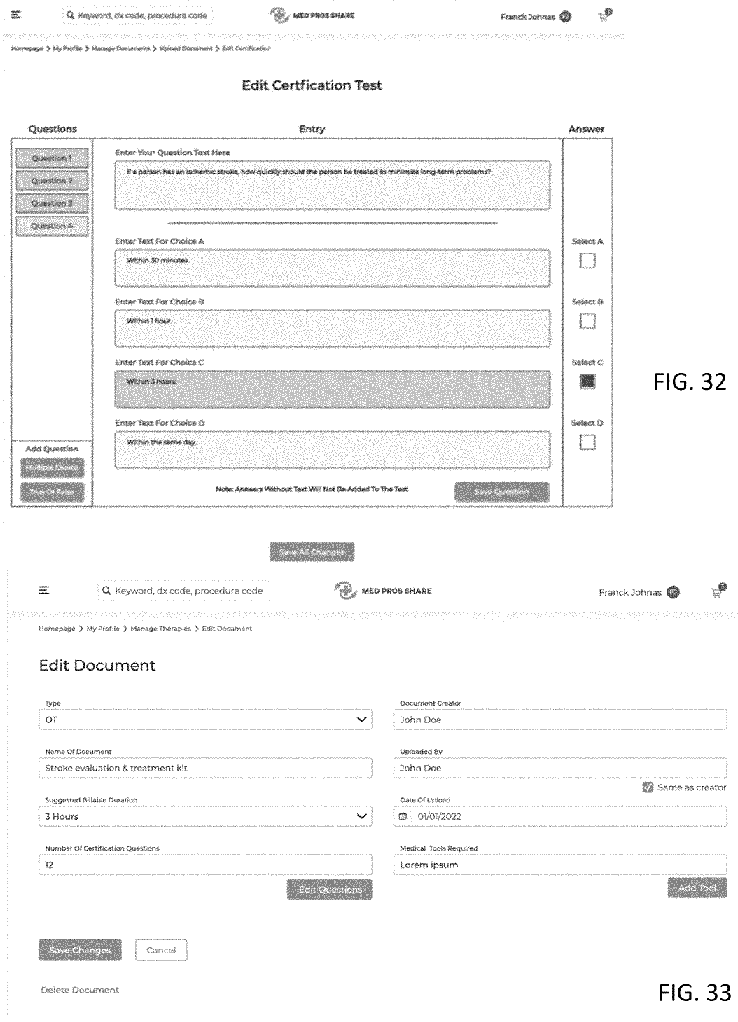

A fourth part of the upload comprises value media directed to CE credits (continuing education credits) 338. In some cases, such as when the value media is in the form continuing education, templates can be uploaded here to award participants a document to show as proof that the continuing education was completed by the clinician. As part of this aspect, the web system can be operable to monitor progress of clinicians through the continuing education, or sporadically administer short quizzes or surveys to assure the clinician is sufficiently participating in the CE program. In one step for example (FIG. 1), CE test questions are input and saved to memory in the web system along with the correct answers 340. This information is uploaded using for example, Google forms, to memory such as in the database server. FIGS. 30 and 32 and depict one example of a wireframe whereby test questions are created and edited. Scoring requirements 342 for the CE test can also be uploaded and saved to memory to assess user's competence in relation to the test questions.

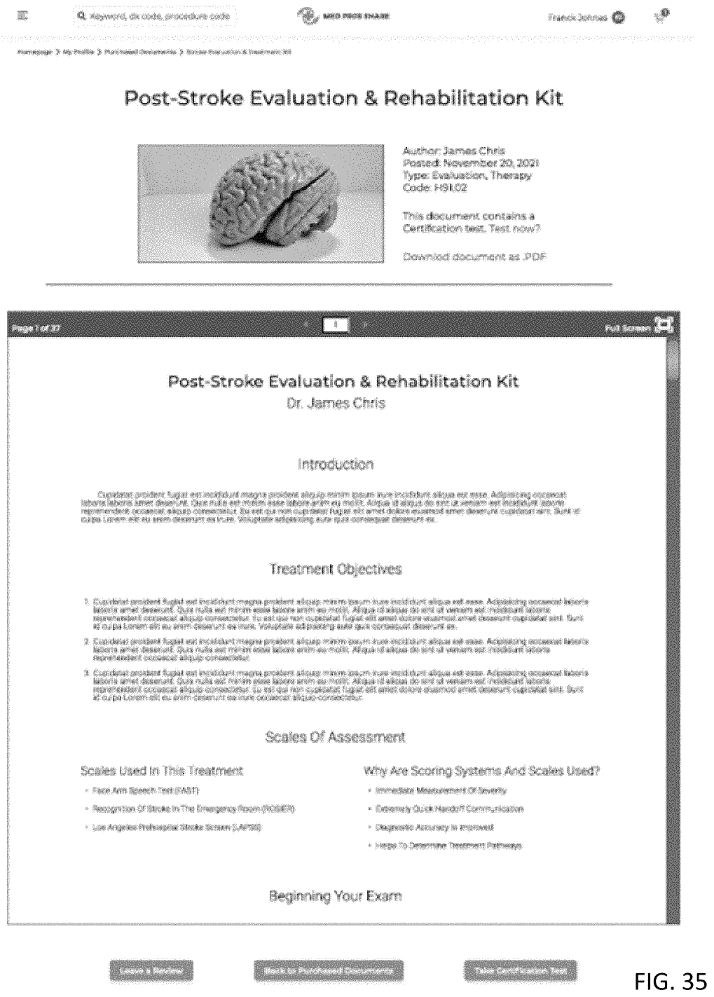

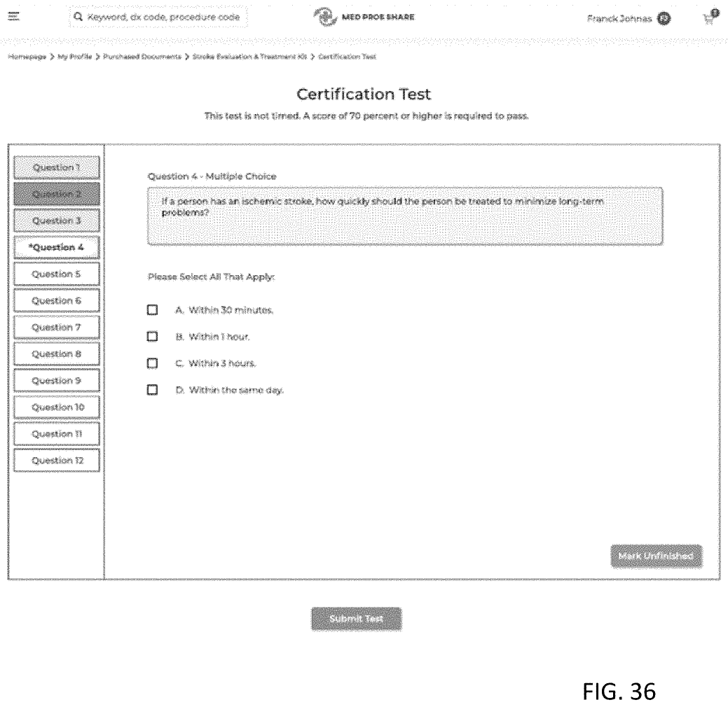

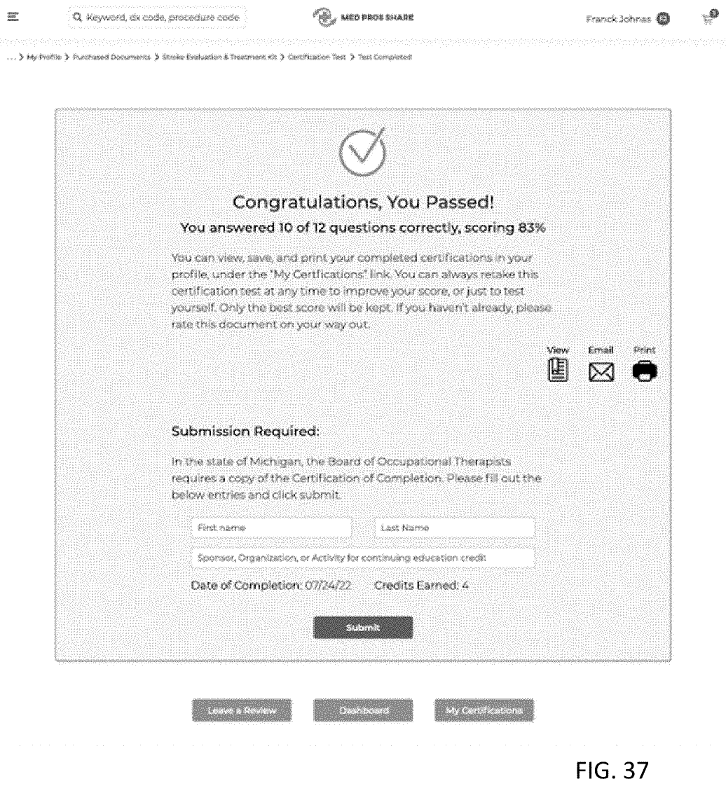

An example of steps for providing CE testing and distributing credit 362 are depicted in FIG. 5. In addition, an example of a wire frame depicting a first test page related to post-stroke evaluation is depicted in FIG. 35. In a first step of CE testing, questions previously uploaded are displayed to a user on a client computer terminal 132 for administering the CE test 364 and the user inputs their answers by way of a computer input device as noted by the example wireframe in FIG. 36. In a following step, the test is scored 366 whereby a computing system within the web system utilizes a processor to compare the users answers to the correct answers 340 that were previously loaded. Upon meeting the scoring requirements, a certificate of completion 368 is prepared by the web system and a copy of the certificate is saved as a continuing education certificate for validation 369. An example wireframe to illustrate passage of an exam is illustrated in FIG. 37. The page can include further user facing options that give the user the means to submit the certificate to an interested party such as a state licensing board.

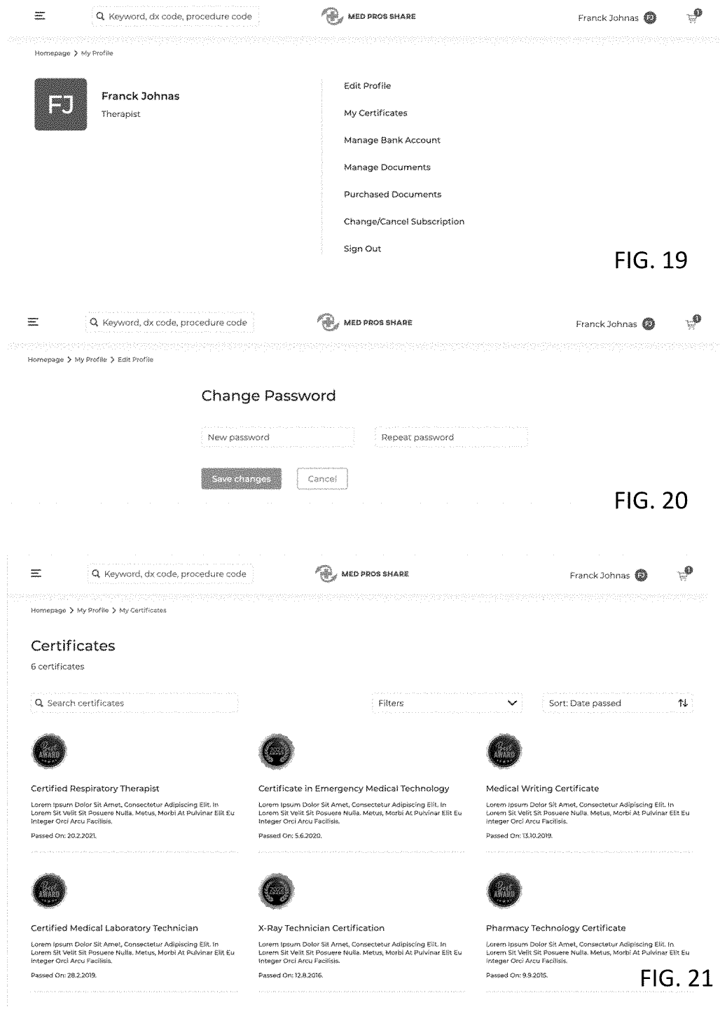

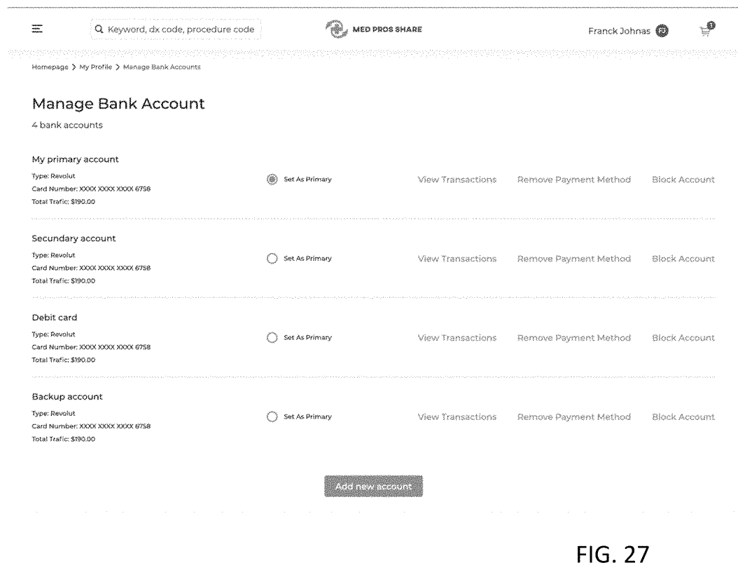

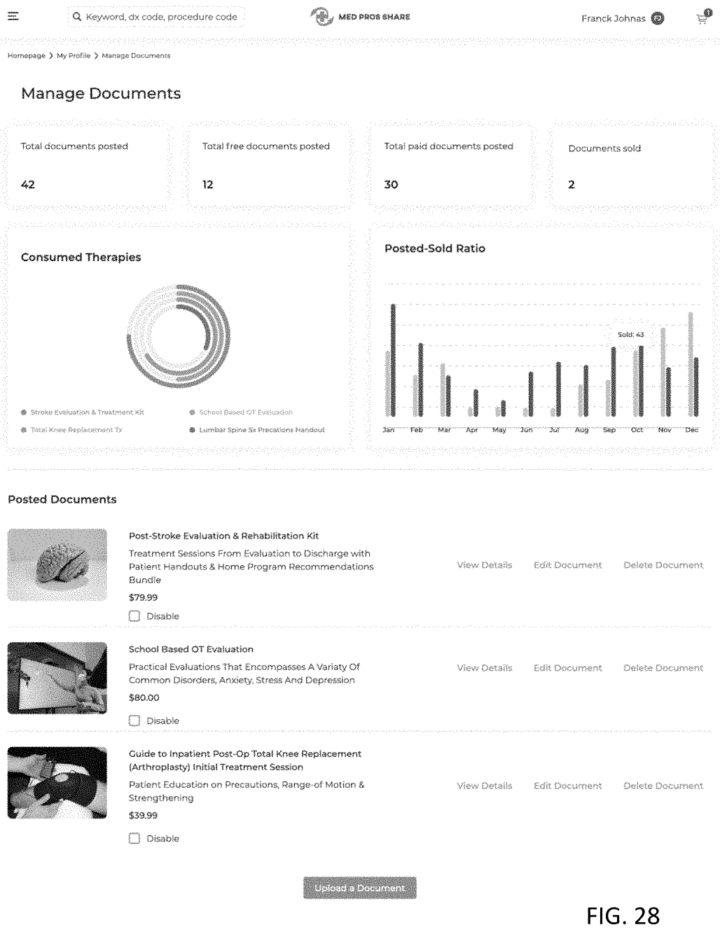

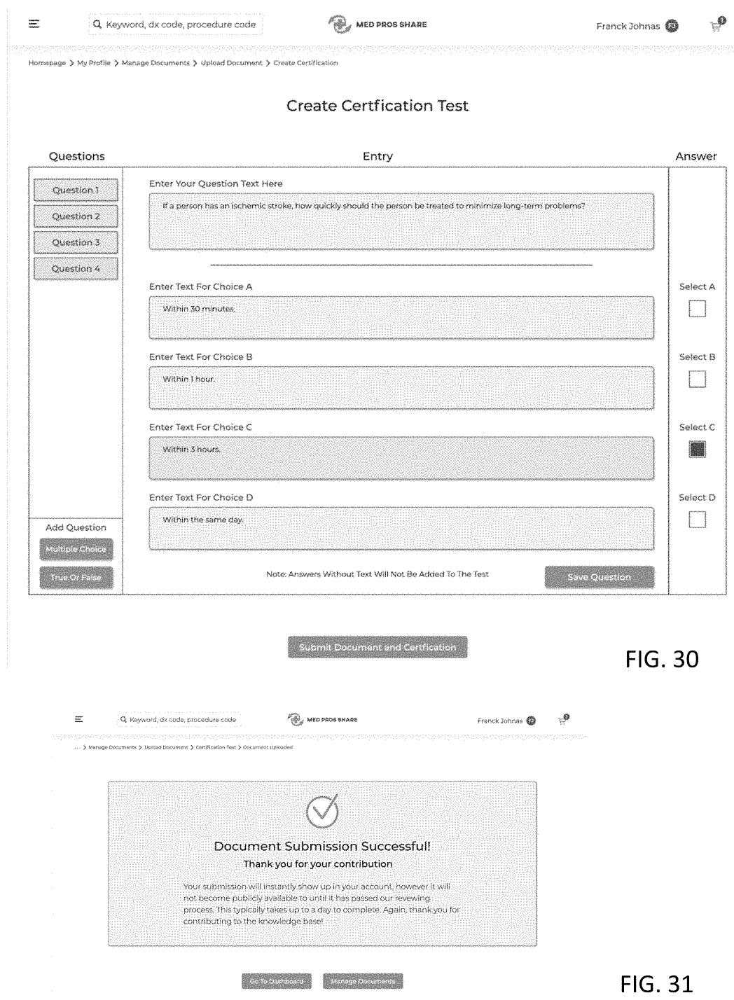

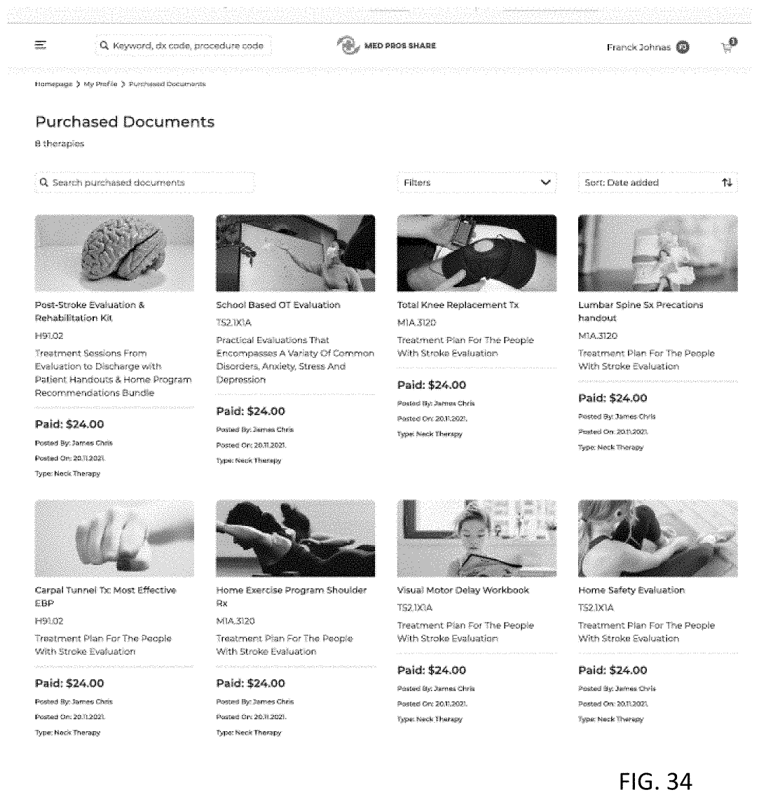

Other wireframe examples of webpages that can be utilized in the web system include FIG. 19 depicting user facing options and information related to a user's profile. From this page, a user has access to many other areas by mouse clicking those listed in the figure. FIG. 20 is a wireframe example of a page utilized to change a user's password. FIG. 21 is a wireframe example of a page depicting various certificates a user has earned through CE testing offered by the web system. These certificates can be utilized to demonstrate proficiency in the areas listed. FIG. 31 depicts a wireframe of a webpage showing an effective value media submission whereas FIG. 33 depicts a wireframe example of a page utilized when wishing to edit the value media which in this case is a stroke evaluation and treatment kit. FIG. 34 is an example wireframe of a webpage depicting various documents that have been purchased by either a general public user or medical professional user of the web system. The page displays information such as cost, when posted, and a description of the value media. FIG. 27 is an example wireframe of a webpage depicting various financial accounts that can be utilized to make or receive payments related to value media or other medical products sold on the web system. FIG. 28 depicts an example wireframe of a web page used in the web system to manage uploaded value media documents for a user logged into the web system. The page shows posted documents, posted/sold ratios, total documents posted, free documents posted, paid documents posted, and quantity of documents sold. The page also has user facing options to view the details of, edit, or delete the posted value media.

An additional segment of onboarding 330 is the establishing proof of intellectual property ownership 344. In one embodiment, establishing proof of intellectual property ownership comprises the step of the contributor uploading relevant trademark ownership information such as a trademark registration granted from the U.S. Patent and Trademark Office, or proof of registered copyright at the registration portal of the copyright office. This information can be self-validated whereby the contributor validates by electronic signature that the uploaded proof of intellectual property is authentic and valid, or validated by administrators of the web system who receive the intellectual property documents. In an alternative form, the proof is in the form of a signed oath or declaration. For example, a declaration signed with the legal name of the author, would identify the value media being uploaded, and include a statement such as for example: “I hereby declare that I am the original sole author or original joint author of the attached media. I further declare that I (we-in the event of joint authors) retain full ownership including copyrights to the authored work and have no obligation to assign ownership of the media to a current or past employer or other interested party. I hereby acknowledge that any willful false statement made in this declaration is punishable under 18 U.S.C. 1001 by fine or imprisonment of not more than (5) years, or both. In addition, I declare that all media uploaded to this site is clearly marked with copyright symbols and where appropriate trademark insignia indicating ownership and source of the material.” This proof of intellectual property ownership can be in the form of an uploaded document, or by having the contributor read a similar passage and confirming the terms by onscreen clicks from a user facing option.

As noted above, in preferred embodiments, the contributor makes an oath that they are the original sole or joint author of the value media and that they have no obligation to assign rights to the value media to a third party. This addresses work completed for example, by a contributor while employed by another party or when paid or commissioned to create the work by another party. Original works are works created by the individual(s) signing the declaration and not derived in part or whole from work done by another party. Therefore, the works cannot simply be improvements of another person's work. In the event the user is absent intellectual property rights, user facing options include links to legal entities 346 such as Legal Zoom® or links to trademark attorneys that can be utilized in partnership with the web system through an affiliate program or something of the like to establish these IP rights. Once the appropriate intellectual property rights are obtained, the user returns to step 344 to establish IP proof.

Once the value media is uploaded and proof of intellectual property ownership is established, a user facing option includes the creation of the contributor's value media store, a hyper-link to the value media, and creation of the contributor's dashboard 370 to monitor metrics and other information related to the contributor's uploads. The contributor can then be faced with user facing options designating the type of media/document and the intended audience 372. For example, from the user facing options on a type of media page, the contributor can choose the intended audience. The audience can be ‘General Public’ whereas the media is educational content and targeting skill development of the general public. Alternatively, the audience can be intended for ‘Licensed Medical Professionals’ such as but not limited to, physical therapists (PT), occupational therapists (OT), or speech-language pathologists (SLP). Such media for this audience can include for example, treatment plans and evaluation guides intended for various medical conditions. User facing options can also include designating the setting 374 for which the media is intended, such as but not limited to for example, outpatient, inpatient, mental health, school based, preparation media for a corresponding Board exam, or other. Further types of document options include for example, the selection of ICD-10 diagnosis codes, ICD-10 procedure codes, and an intuitive therapy name for the value media. In each of these cases, the fields can be prefilled by the software by AI recognition of the contents of the document. As yet another user facing option, the user can then input a title/name for the value media 376. At the end or during the onboarding process, the value media and any other uploaded materials can be reviewed for appropriate content 378.

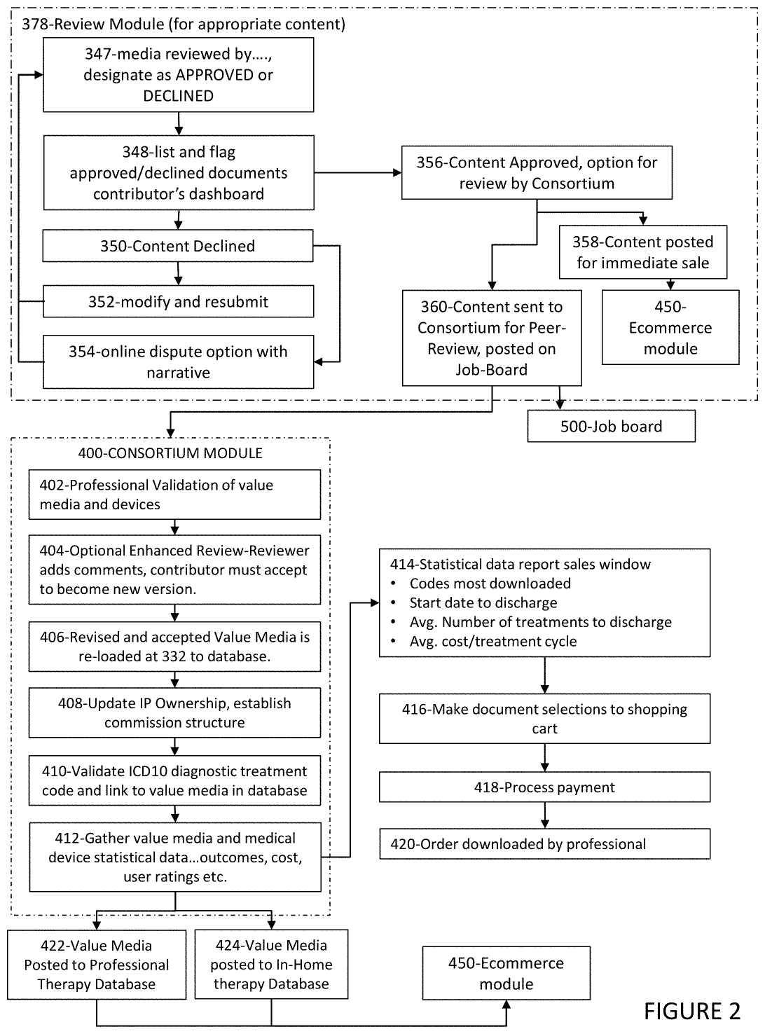

In one embodiment, a process for reviewing appropriate content 378 is depicted at the top of FIG. 2. Onboarded value media is assigned and reviewed by one or more competent reviewers who designate within the web system the value media as Approved or Declined in relation to preset standards 347. The reviewer activates user facing options causing the value media to be listed and flagged as approved or declined 348 on the contributor's dashboard. If the content of the value media is declined 350, the contributor of the value media is notified on their display and given the option to modify the value media and resubmit it 352, after which it is once again reviewed and designated as approved or declined 347. Alternatively, rather than modifying and resubmitting the declined content, a contributor can submit through their user facing options an online dispute with a narrative 354 supporting why the value media should be approved. The online dispute is then sent back to the value media review step at 347. Value media that has content approved 356, has an option for posting for immediate sale 358 in the ecommerce 450 module of the web system where it can be purchased by others. The contributor also has the option of sending the value media to the consortium for peer-review 360 after its content has been approved. In this case, the value media is posted on the Job board 500 and made available to those interested and qualified to peer review the material.

In most cases, the information provided in the value media is utilized in the treatment of patients suffering from a physical or mental ailment. As a measure of quality assurance, in some embodiments, the web system includes a consortium 400 module (FIG. 2) that coordinates the steps of final review of the value media. A consortium is made up of consortium professionals having expertise in the discipline or disciplines associated with the value media. The consortium can include active or retired professionals of professional practice, or other qualified researchers or educators having a predetermined level of expertise. Opportunities to participate in the consortium for peer review can be posted on a peer-review job board 360 that is part of the web system described herein. The previously onboarded value media 332 is made available to the peer-review job board and is then distributed to the consortium professionals based on their education, and professional experience to undergo professional validation of the value media and of any complementary devices. Examples of complementing devices may include exercise accessories such as resistance bands, splints, electrical stimulation units, diabetic footwear, specialized tapes, and assistive devices such as canes or walkers. Members of the consortium will then validate the value media and any associated devices 402 to assure they are safe, current, beneficial to the patient, and within the practice standards of the selected clinical discipline.

In some cases, the value media will undergo an optional enhanced review 404 whereby members of the consortium modify or enhance the value media to improve its value and/or effectiveness. For example, the value media can be edited at this step to remove portions of the media that are considered outdated practice, enhanced to clarify vague descriptions, add proprietary treatment methods, or upgraded to utilize more advanced presentation techniques. If approved by the original owner, the revised value media then replaces the previous version and once again onboarded at step 332. In some cases, the enhanced review 404 will lead to members of the consortium to be added as joint owners of the value media relative to their contribution. Therefore, an update to the intellectual property ownership established at 408 is required. Also, it is at this step that commission structures based on sales of the value media are established between original contributors and members of the consortium that added intellectual property to the value media. This sharing of commissions with members of the consortium can be pre-selected, negotiable, or flat percentage or flat rate.

Upon validation by the consortium of the content of the value media and approval of complementing devices for use in associated treatments, the value media and devices are ready for posting within the ecommerce module 450 and viewable on displays to either the professional clinician database of the database server system 130 for purchase by member clinicians of the system at a back-end of the web system, and/or for purchase by patients of member clinicians also at the back-end of the web system.

However, before posting the value media or devices for sales, the consortium validates the assigned ICD-10 (International Classification of Diseases 10^{th} revision) codes to the value media and associated medical devices 410.

The consortium module 400 (FIG. 2) is also utilized to gather and process statistical data on therapeutic and other clinical treatments. This can include data pertaining to the associated medical devices, patient outcomes, cost of treatment, user ratings, most used ICD-10 codes, start date to discharge days, average number of treatments to discharge, and average cost per treatment cycle, etc. This data is gathered 412 and can be offered to interested parties such as clinicians, insurance companies, and others by display in a statistical data report sales window 414 and made available for access at the back end of the web system. The data can be offered as a courtesy or with payment of a one-time fee or a subscription and then downloaded from the web system.

The facing options on a screen of a computing device of the web system includes making document selections 416 for purchase of the statistical data at the statistical data report sales window 414 discussed above. Desired statistical data is selected or can also be provided via upload to the consortium by a group, business, or corporation for statistical review and analysis by the consortium in the form of de-identified data. For example, a therapy clinic may provide a specific ICD-10 diagnosis code and the corresponding treatment codes with a specific set of identifiers, such as time of onset to time of discharge. The results are compiled based on selections in the statistical data report sales window 414. From that point, document selections are added to a shopping cart 416. Payment information is collected, and the payment is processed 418. The web system then grants the purchaser access to the chosen statistical data where it is downloaded by the purchaser. Alternatively, selected pieces of the statistical data can be displayed as a courtesy to prospective purchasers of the value media and associated medical devices for the purpose of assisting the purchaser with their value media and medical device choices. For example, a therapist in a rural community in the U.S. or a foreign country such as India, may be unfamiliar with rehabilitation techniques after ankle replacement surgery and lack experience in creation of a treatment plan. However, the rural therapist can search the database of the web system and identify value media directed to rehabilitation post ankle surgery. The transference of knowledge can be promoted and monetized for the organization that paid the consortium to compile and analyze the data and can then post the results for sale to further share and monetize the research findings and their investment into research. This information is further validated by reviewing displayed statistics regarding most downloaded value media and highest rated value media, the prospective purchaser is guided to highly rated post ankle surgery rehabilitation programs. In preferred embodiments, the web programs are translated into various languages used about the world.

Once the consortium work is complete, an administrator of the web system changes permissions associated with the value media thereby providing access facing options to unlock the value media to clinicians, clinic owners, and patients from the backend of the web system. Value media saved to the Professional Therapy (clinical) database 422 is then available for ecommerce purchase at the website by clinicians of that discipline and may be paired with applicable products/devices for clinician use. The value media can be filtered by user facing options to determine, for example, what would be the best treatment code given a specific diagnosis for time to discharge. Value media saved to the In-Home therapy database 424 is available for ecommerce purchase to patients to access their home programs, find value media of interest, and for purchase of associated medical devices if necessary. This is particularly useful for rural clients whereby travel distance limits the frequency of face-to-face encounters between the healthcare practitioner and patient. The presence of readily available value media to support treatment ensures the best home care outcomes.

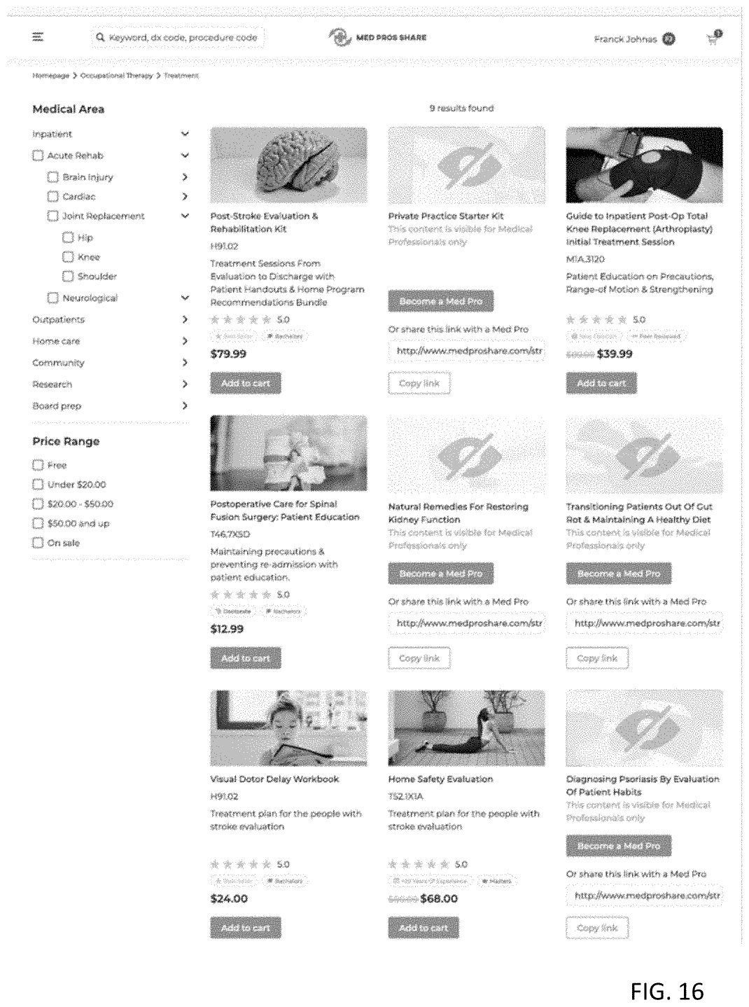

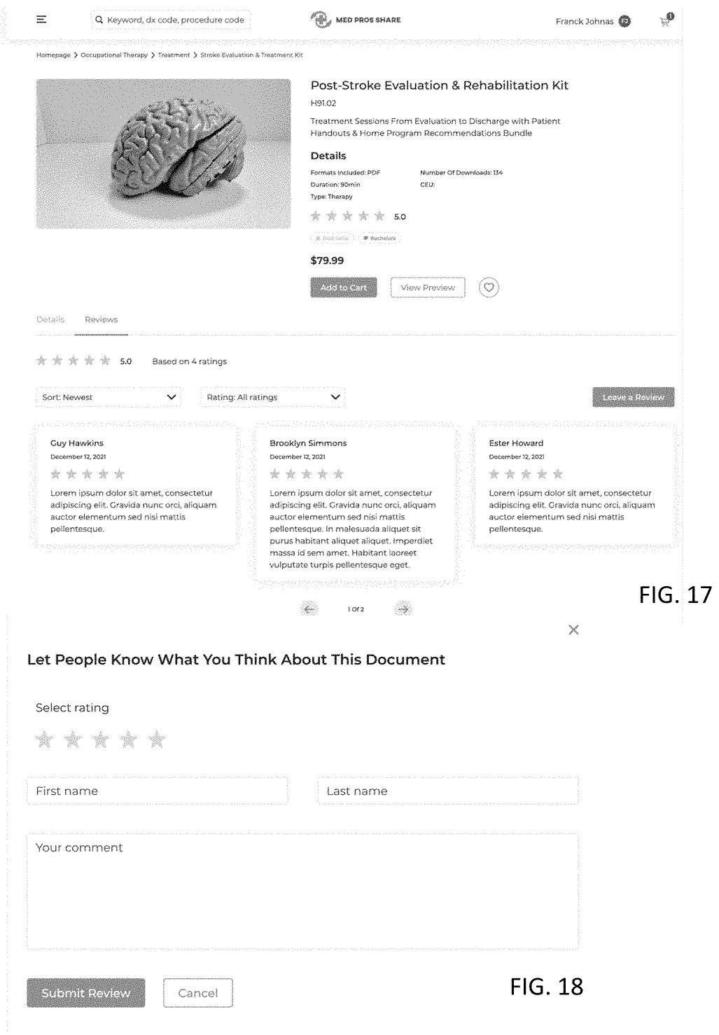

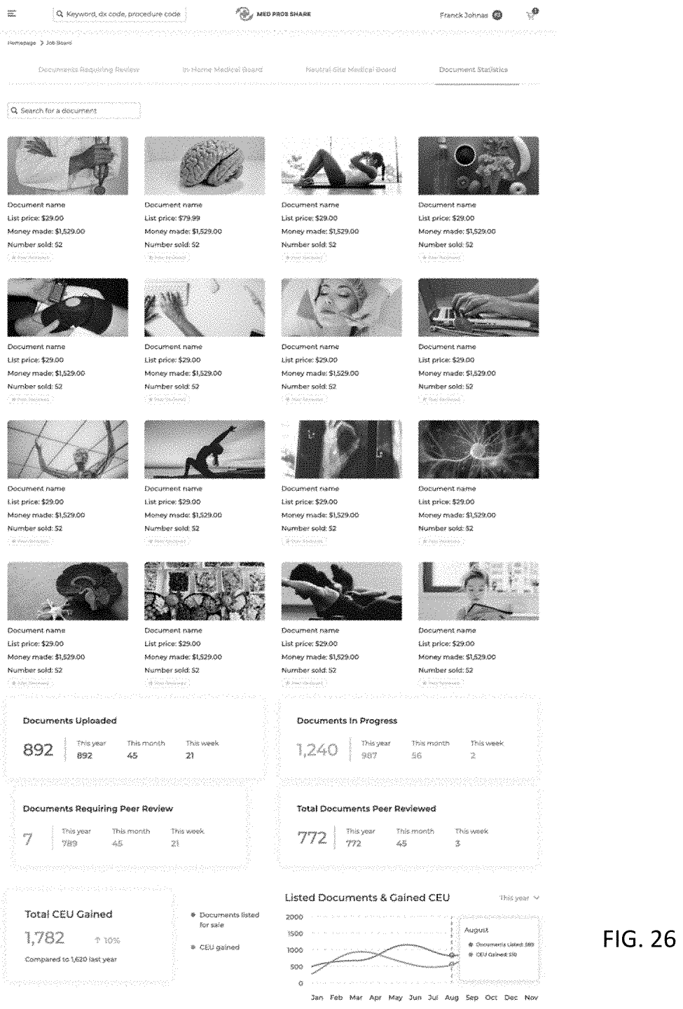

At the back end of the web system in an ecommerce module 450 (FIG. 4), user facing options from a client computer terminal 132 display are presented to those interested in purchasing the value media and associated medical devices. The process at the backend in the ecommerce module begins with establishing a purchase account 452. At this step the purchaser selects through purchaser facing options to classify them as a clinician, a patient, or other individual associated with a designated business. Login information is chosen which typically can include a user-name and password. The purchaser then logs into the purchase account 454. In some cases, a previous log in such as log in at 322 can be used to gain access. Otherwise, new purchaser facing options then appear on the purchaser's display. These options can vary according to the purchaser's classification as a clinician, a patient, or other designation such as a business. It is here the purchaser is presented with options to shop the various value media and medical devices. Search windows can be utilized to search value media options by filters such as treatment type, keywords, and diagnostic or treatment codes 456. At this point the purchaser interacts with options on their display to make value media selections 458 using input devices. FIGS. 16 and 17 depict an example wireframe of value media available for purchase whereas the bottom of FIG. 26 depicts statistical data related to the documents including the total number of documents uploaded, documents in progress, documents requiring peer review, total number of documents peer reviewed, number of documents listed for sale, and total CEUs gained. In some embodiments, the value media is saved with a generic skin such as for example, a generic medical rehabilitation theme, or a skin consistent with the logos and branding of an associated clinic. In other embodiments, the purchaser is presented with purchaser facing options that includes selection from a variety of skins 460. An example of these skins can be thought of similar to a clipart library that applies a variety of images based on the client, business, or clinicians searched preferences, such as a Christmas theme or a preferred activity to a treatment plan. Examples include but are not limited to applying a golf theme to a shoulder rehab program or billiard balls applied to targets for a patient who is struggling with a visual field deficit following a stroke. This allows for the customization of treatment strategies while decreasing the replication of the similar treatment strategies by just applying different themes keeping the database information dense. The purchaser then selects a desired skin to apply to the value media and saves these selections in an electronic shopping cart 462. Skins are provided at an additional cost and added to the initial download. Typically, they is a fee that allows clinicians to easily change the skin for a low cost and own that document for clients with the same interest in the future.

In some cases, the purchaser will have facing options from their display to purchase a medical device that complements or is an adjunct to the value media. The purchaser will make on screen selections for the linked medical device 464 and place it in the shopping cart. Further purchaser facing options will include processing payment 466 which will include any variety of purchasing options including ‘pay later’, and various payment routines known in the art such as credit card, debit card, gift card, EFTs, and online payment systems such as PayPal. The purchaser is then presented with a receipt for purchase which can be saved on electronic memory or printed by way of an associated printer. The purchaser is then presented with facing options to download the value media in the account holders individualized library of downloads for easy access to a memory device, have it sent to an email address, or immediately print the information as part of order fulfilment 468. At this point, the purchaser can log out of the purchase account or will be timed out by the web system software 470. Once payment is processed 466, the web system will calculate the predetermined commissions and initiate payment to each contributor 472. If the purchaser ‘pays later’, the web system can alternatively delay the payments to contributors until after payment is complete from the purchaser. A portion of the sale may be used as revenue to the web system owner.

Referring back to step 464 whereby the purchaser selects devices that are linked to the value media. In some cases, the device order with payment is sent to the manufacturer 476 or other 3^{rd} party supplier. The manufacturer will then fulfil the order 478 by sending the device to the purchaser's home or another location such as directly to a clinic where the purchaser is receiving treatment. In some cases, the manufacturer or other 3^{rd} party supplier will provide a commission 480 to the owner of the web system and/or owner's of the associated value media.

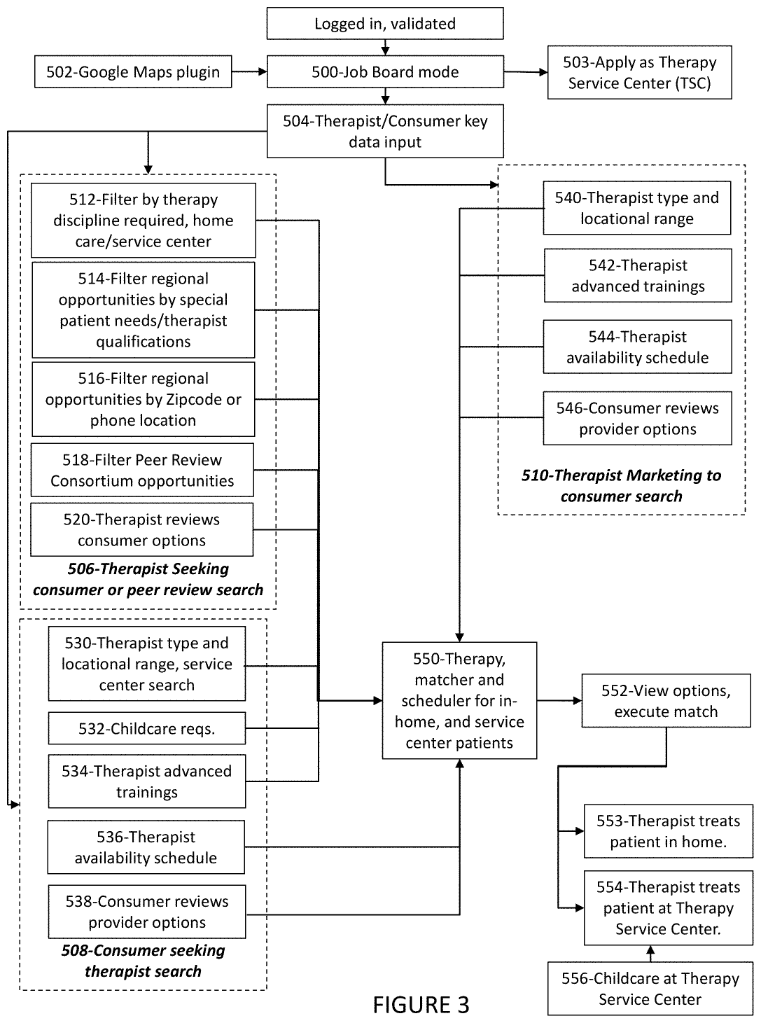

FIG. 3 depicts a job board module of the web system. As a user facing option on the display of a client computer terminal, a user can enter the job board 500 from a variety of different pages in the web system as previously indicated. The job board primarily addresses 3 needs related to matching from a database, medical professionals seeking to provide treatment with consumers needing their services.

The first need is for medical professionals such as therapists (i.e., PT, OT, SLP) seeking opportunities to provide respective medical treatments to individuals (consumers) needing medical care or for medical professionals seeking opportunities to perform peer reviews 506. The second need the job board addresses is for medical professionals wishing to market to consumers. The third need the job board addresses is for consumers seeking medical professionals. In the case of matching medical professionals with consumers seeking treatment, therapy sessions can occur within the patient's home or within a Therapy service center (TSC), or other agreed location. Using demand analysis based on demand for therapy in various locations, a user of the web system can apply to establish a Therapy Service Center (TSC) 503 which can be a cooperative space used by therapists and perhaps other medical professionals to provide therapy services to consumers. Such a space provides a treatment area for utilization by therapists who wish to practice independently or by medical professionals who wish to practice on a part-time basis but are otherwise without a facility to treat patients. The TSC can be financially supported by a flat or percentage rate with each encounter at the TSC, or by other arrangement such as a flat monthly rate.

In preferred embodiments, the job board 500 utilizes a mapping app plugin 502 such as Google Maps to match medical professionals and potential clients within a specific geographic location. In each of these scenarios, the process begins by having the therapist or consumer (potential patient) input key data using an input device to be saved in memory of a database in the web system 504. For example, for a consumer, this information can include data such as name, address, phone number, diagnosis, therapy sought, schedule availability, start date, and details regarding budget, etc.

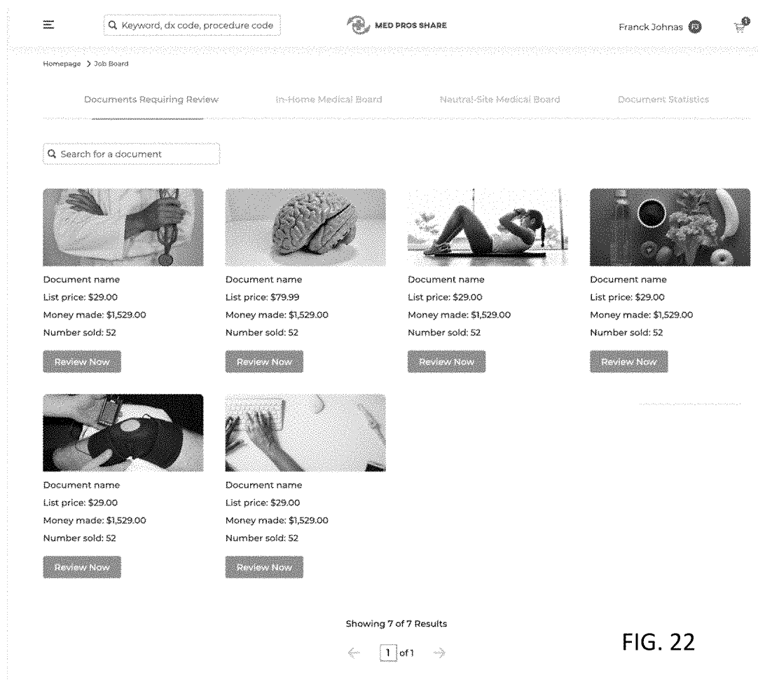

Assuming the medical professional is a therapist in this example, the job board module utilizes one or more of the following steps to match therapists seeking a consumer needing therapy or for a therapist seeking a peer review opportunity. These steps are displayed as user facing options on a client computer terminal 132 of the web system. For example, the user may encounter a selection that sorts through consumers seeking therapists. Various filters can be applied such as type of therapy required and whether the therapy need is for home care or can be performed at a service center 512. Another filter may isolate consumers according to special patient needs or special qualifications of a therapist 514 (i.e., cardiac rehab certified). Another filter causes a display of regional opportunities identified by zip code or phone location 516. When searching for a peer review consortium opportunity, the filter finds opportunities matching the expertise of the medical professional 518. Once the preferred filters are applied, the therapist is then able to review consumers meeting the filtered requirements 520 or peer review consortium opportunities. These filtered results can then be loaded into a therapy matcher and scheduler 550 for both in-home and service center patients. FIG. 22 depicts a wireframe example of documents available for peer review within the job board. In this case, the medical professional would click on the ‘review now’ icon to engage in the review of the document.

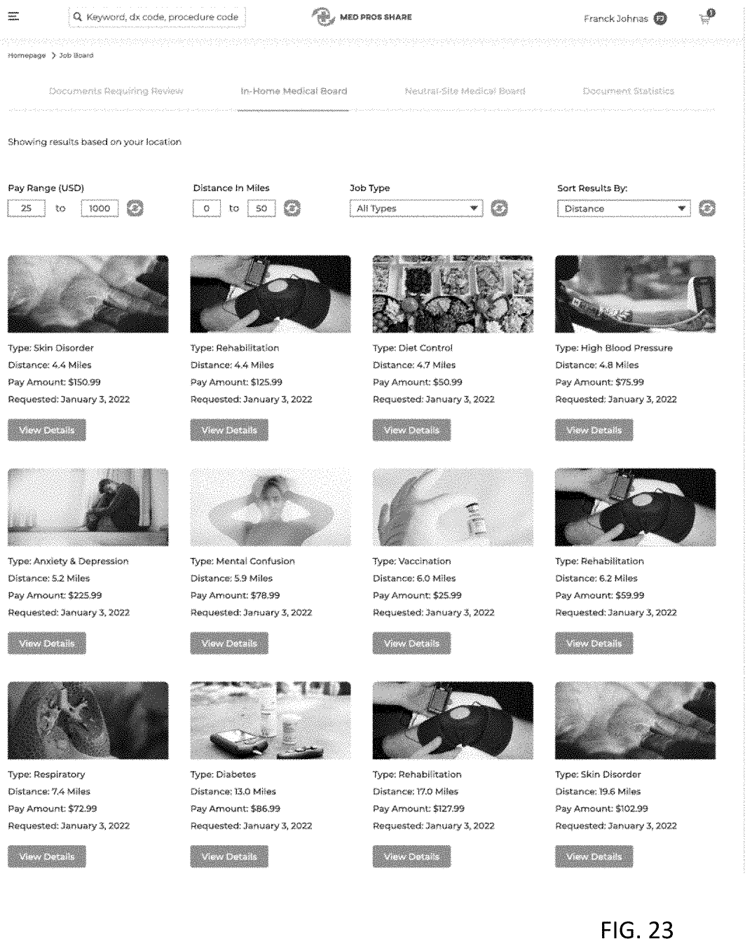

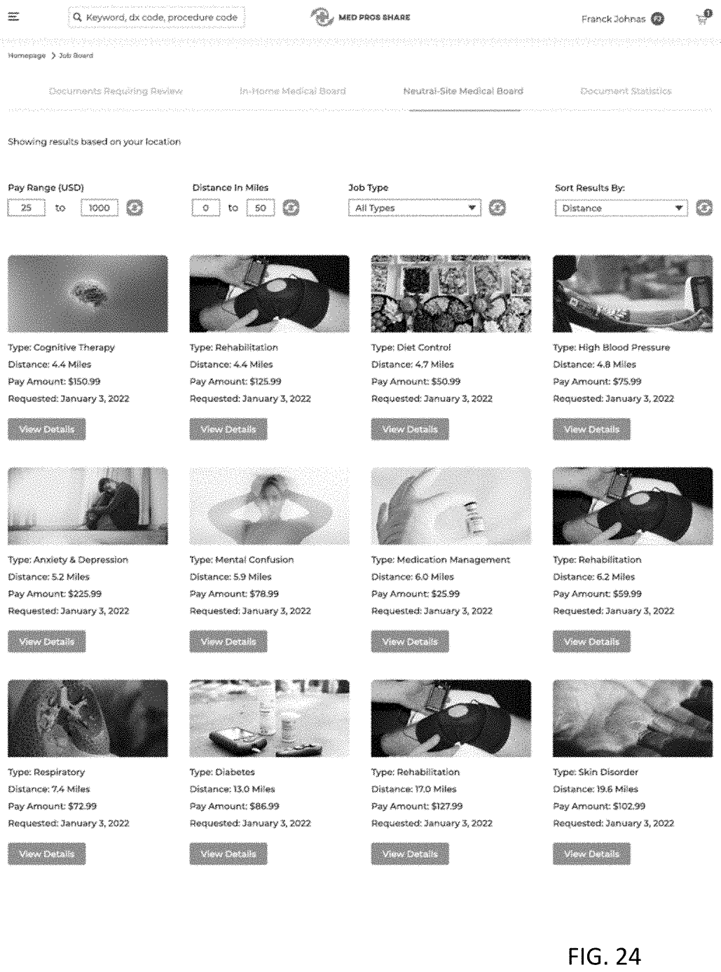

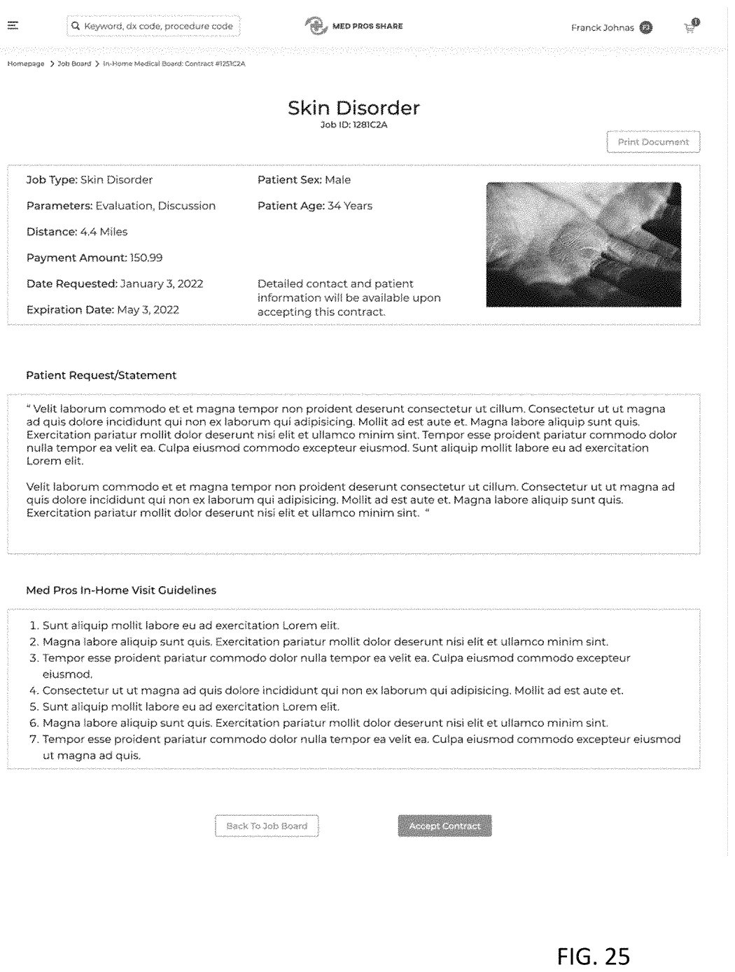

FIG. 23 depicts a wireframe example listing various medical treatment opportunities posted by consumers seeking medical treatment from medical providers. Note in this example, ‘type’ or ‘diagnosis’ is listed along with the distance away from the logged in medical professional, the amount the patient is willing to pay, and a date for when the service was requested. Noted also are the filters at the top of the page for pay range, distance, job type, and filters to sort the results. FIG. 24 is a similar depiction; however, these results are filtered to show medical treatment sought by consumers at a ‘neutral-site’ such as at a therapy service center. The menu at the top of the wireframe provides the user the ability to select between these options (i.e., documents requiring review, In-Home medical board, neutral-site medical board, document statistics). FIG. 25 depicts an example wireframe of a webpage providing details of a medical need when the ‘view details’ icon is activated. At the bottom of the page is an ‘accept contract’ button that accepts the match between the medical professional and patient. Latin is utilized in some portions of the wireframes for demonstration purposes to illustrate location of text.

Similarly, the job board module can be utilized for assisting consumers find available therapists 508 to provide treatment. For example, a consumer logged into the system job board module utilizes one or more of the following steps to match consumers seeking a therapist. These steps are displayed as user facing options on a client computer terminal 132 of the web system. A filter is applied to find a local Therapy Service Center or find therapists within a specific geographic range 530. Other filters may include whether childcare is available 532 at the treatment location. This filter is particularly useful for consumers with children and who would be unable to attend a treatment session without childcare. Another filter can be to isolate therapists having specific advanced skills or training 534. Yet another filter option can be based on therapist availability 536 to find a therapist that can meet the schedule demands of the patient. Upon filtering down the options, the consumer then reviews which providers are available. Again, the filtered results can then be loaded into a therapy matcher and scheduler 550 for scheduling both in-home and service center patients. Through this system of matching, the therapist then can view matching options and execute the match 552 which also includes the patient also accepting the match. The therapist (medical professional) can then proceed to treat the patient in their home 553, or at a designated Therapy Service Center 554 with the option of having childcare at the service center 556 if so equipped and needed by the patient. The available childcare can also be helpful to the therapist who otherwise is unable to work unless childcare is available. The flexibility of this system encourages and enables both healthcare professionals and consumers seeking care to easily find matches between their schedules. Retires and stay at home parents will be attracted to the flexibility and function of this system thereby attracting them back to the workforce.

The job board module can also be utilized by medical professionals to market themselves to consumers. In this case, instead of a therapist searching through postings by consumers seeking treatment, the therapist markets themselves to consumers 510 on the web system. In this module, the therapist will encounter user facing options that allow them to select the type of medical professional/therapist that they are, as well as a locational range in which they are available to operate 540. Other user facing options provide for the selection of advanced therapist trainings or certifications that the therapist has 542, and selections to indicate their availability 544 within the systems therapy matcher and scheduler 550. All of this information is available for display in addition to consumer reviews of the provider 546 to consumers shopping for a good match to therapists who are available. FIG. 17-18 depicts a wireframe example of a review page that can be used to provide ratings of various value media that has been uploaded. A similar page can be configured to provide reviews of medical professionals.

The data captured by in steps 506, 508, and 510 is processed and displayed in the therapy matcher and scheduler for in-home and service center patients. An initial invite is executed by one of the parties (medical provider, patient) that includes a date, time, and place for the first appointment. The match is executed, and the first appointment is set 550. At this point, the therapist treats the patient in their home 553 or at a therapy service center 554 at the accepted date and time. If available, childcare is made available at the therapy care center 556 for the benefit of the patient seeking treatment, or for the benefit of the therapist needing childcare in order to work outside the home.

The web system disclosed in some embodiments herein is particularly useful for patients and therapists that have limitations in their schedules. For example, for therapists or patients who can only work between the hours of Sam-9 am. The web system intends to not only facilitate the needs of patients seeking help from therapists, but it also facilitates the needs of therapists seeking opportunities to provide patient care. Unlike the traditional model of care that is often rigid in timing and in location, the web system matches medical professionals directly with patients in their home or within a flexible environment such as a cooperative therapy center. The web system also intends to encourage the upload of useful value media that can be distributed to others. In some embodiments, utilizing the job board 500 requires the therapist to upload value media such as an evaluation form, exercise program, therapy skills, or educational pamphlet before scheduling and initiating a first visit with the patient. Uploading of value media such as this is depicted at 505 (FIG. 1). This action additionally counts as continuing education and can earn the contributor CEUs. In other embodiments, this condition is waved thereby allowing the therapist to see the patient without an upload of value media.

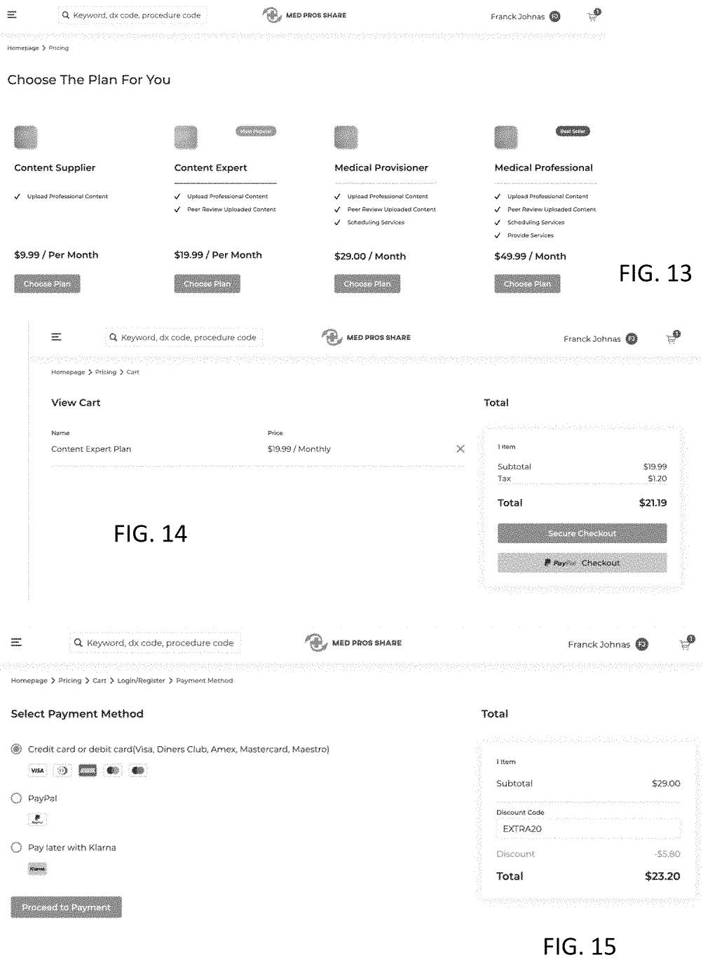

Upon arriving at a landing page of the web system, the user has a user facing option to establish a professional account 320, or to establish a general public account 302. The general public account is for use by members of the general public who use the web system to either seek and arrange one or more treatment sessions with a medical provider logged on to the system, to access home therapy programs assigned by the therapist or medical professional, to use the site to find and purchase value media, and/or to use the site as a means to advance their knowledge and skill through CE testing which can in turn earn them CE credit. In some cases, a medical professional may register using a general public account to simply access the value media available on the site. FIG. 13 illustrates one embodiment of various plans available to a professional account user, however the plans and the costs can vary with each tier adding functions. As illustrated here, a professional account may be simply a ‘content supplier’ that uploads value media for sale on the site. A ‘content expert’ can also peer review uploaded content for pay or to earn CEU credits or both. A ‘medical provisioner’ can also use the scheduling services to locate and schedule patients seeking their healthcare services. A ‘medical professional’ can also advertise their services to other medical professionals. Once a plan is selected by the user and added to an ecommerce cart as illustrated in FIG. 14, a payment method is selected, and payment is processed as illustrated in FIG. 15.

In one embodiment, a process for establishing a general public account comprises the user being faced with an establish general public account icon as a user facing display option. Upon activating, the user enters some basic information about themselves to establish a general public account 302. Once the account is established, the user is faced with a new contributor facing option to log on to the site 304. This may include for example the input of a username unique to the clinician and a password. Once logged in, the user is presented with several additional user facing options. One option is to be transferred to a general public dashboard 600 which is a page containing a variety of information such as depicted in FIG. 7 applicable to their use of the system. Metrics for this information can be constantly displayed on the dashboard or displayed on clicking an icon. As noted, the items 640-658 mirror items in the medical professional dashboard 602. However, in the case of the general public dashboard, some of the displays are greyed out and not operable since they may not apply to a general public user. However, although ghosted, they do provide the user with a sense of the system's capabilities a perhaps encourage upgrading in some cases to a medical professional account. Another user facing option after a general public user logs on to the site is to enter a filtering area where the user can choose a particular medical discipline(s) that they are interested in 306. This selection narrows options for a general public user so they don't become overwhelmed with options since at a following step, they have the user facing option to search and purchase value media 308 posted on the site or alternatively to go to the job board 500 where they can seek and schedule the services of a medical professional as depicted in FIG. 3. Another user facing option after logging on the site is to be transferred to a window displaying home therapy programs 310 a therapist may have designated for the general public user as an extension of their therapy. Alternatively, another user facing option links the general public user to a CE testing area where the user can engage in CE testing on select subjects and obtain credit for it 312. For example, a member of the general public may wish to study material related to “caring for an autistic child”, take a test on the material presented, and receive CE credit upon passing. This may then allow for example, the user to apply for positions in the community requiring this certification. CE testing can also be used to test patients understanding of a home program assigned to them by a therapist thereby increasing the accountability of these patients.

FIGS. 6 and 7 depicts an example of the types of information that can depicted on the display of a client computer terminal 132 when selecting the option to display the medical professional dashboard 602 (when logged in as a medical professional), or to display the general public dashboard (when logged in as general public). As noted, the information displays 640-658 in FIG. 7, replicate the information displays in FIG. 6, however, many of the information displays will be greyed (so they can be seen), but inoperable to allow the general public user see the options available to the professional user in the event they wish to upgrade to be a professional user. As a user facing option, activation of the dashboards causes display of information such as a total count of the value media uploaded by the user 604, a display of the total count of value media peer reviewed the user 606, a display of value media uploaded and for sale 608, a display of the amount of value media versus the amount peer reviewed, expected earnings, and related due dates for material to be reviewed 610, a display of value media listed for sale, compensation to date, and cumulative sales 612, a display of flagged value media that was removed due to inappropriate content, a display of value media in progress, not yet published for sale, with a progress bar 616, a display of value media in progress but not yet ready for sale 618, a display from a CEU calculator of total CEUs earned for peer reviewed and uploaded documents, and a display of total CEUs earned for peer-reviewed and uploaded documents. Since licensure for most health professions requires the medical professional to accumulate CEU on an ongoing basis, these CEUs can be used to maintain licensure.

FIG. 8-9 depicts portions of an example environment for implementing aspects of the web system in accordance with various embodiments. In some embodiments, various modules and structures referenced herein are implemented in software. The software utilizes a processor to execute program code read from a nontransitory program memory store. The program code includes instructions implementing the various modules and structures.

As noted in FIG. 8, example computing environment 100 includes a processor 112, and memory 114 (non-volatile memory for storing program code and volatile memory for storing variables in use by the processor). Computing environment can also comprise a control bus 110 utilized for the processor 112 to use to obtain inputs and provide outputs. One such input/output includes a network interface 124 that may connect to a network 126 via the network interface 124 or alternatively wirelessly via an antenna. Other interfaces include a display interface for interfacing to a display (video device 102), a button interface for interfacing with button inputs/outputs and hardware such as a keyboard input 106, and a touchscreen interface for interfacing with a touchscreen. At times, one or more of these elements are not required.

As illustrated in FIG. 9, computing environments can include but are not limited to personal computers, laptop computers 140, tablet computers 138, mobile computers/smart phones 134, servers 130, embedded computer systems, and similar devices communicating through a network 126. The contributor(s) of the value media, purchasers, and will typically operate from client computing systems. In some embodiments, an administrator computing system 128 is utilized to provide functionality for other client computing systems.

The network can comprise any variety of networks known in the art including but not limited to an intranet, the world wide web, a cellular network, a local area network, a satellite network or any other such network and/or combination thereof. The various protocols and components used to communicate via these networks are well known. Communication via the network can be enabled by wired or wireless connections and combinations thereof. For example, the network includes the world wide web and/or other publicly-addressable communications network, as example computing environment 100 could be part of a web server that receives requests and serves content in response to the requests.

Servers utilized in the web system, may be implemented in various ways. For example, the servers can be implemented as hardware devices or virtual computer systems. In some contexts, servers may refer to a programming module being executed on a computer system. The term “data store” as used herein refers to any device or combination of devices capable of storing, accessing and retrieving data, which may include any combination and number of data servers, databases, data storage devices and data storage media, in any standard, distributed, virtual or clustered environment. The example environment can include any appropriate hardware, software and firmware for integrating with the data store as needed to execute aspects of one or more features described herein. The interface to example computing environment 100 might be entirely over network interface (FIG. 3), such as by having example computing environment 100 implement a web server interface. The handling of requests and responses, as well as the delivery of content can be handled using PHP: Hypertext Preprocessor (“PHP”), Python, Ruby, Perl, Java, HTML, XML, JSON, and/or another appropriate server-side structured language. Operations described herein as being performed by a single device may, unless otherwise clear from context, be performed collectively by multiple devices, which may form a distributed and/or virtual system.

The non-volatile memory and volatile memory (‘memory’, ‘local storage’) can include several separate data tables and other data storage mechanisms and media for storing data relating to a particular aspect of the present disclosure. The environment, in one embodiment, is a distributed and/or virtual computing environment utilizing several computer systems and components that are interconnected via communication links, using one or more computer networks or direct connections. However, it will be appreciated by those of ordinary skill in the art that such a system could operate equally well in a system having fewer or a greater number of components than are illustrated in FIG. 3. Thus, the depiction of example computing environment 100 in FIG. 3 should be viewed as an illustrative example and not limiting the scope of the disclosure.

The various embodiments further can be implemented in a wide variety of operating environments, which in some cases can include one or more user computers, computing devices or processing devices which can be used to operate any of a number of applications. User or client devices can include any of a number of computers, such as desktop, laptop or tablet computers running a standard operating system, as well as cellular, wireless and handheld devices running mobile software and capable of supporting a number of networking and messaging protocols. Such a system also can include a number of workstations running any of a variety of commercially-available operating systems and other known applications for purposes such as development and database management. These devices also can include other electronic devices capable of communicating via a network.

In embodiments utilizing a web server, the web server can run any of a variety of server or mid-tier applications, including Hypertext Transfer Protocol (“HTTP”) servers, FTP servers, Common Gateway Interface (“CGI”) servers, data servers, Java servers, Apache servers, and business application servers. The server(s) also may be capable of executing programs or scripts in response to requests from user devices, such as by executing one or more web applications that may be implemented as one or more scripts or programs written in any programming language, such as Java®, C, C# or C++, or any scripting language, such as Ruby, PHP, Perl, Python or TCL, as well as combinations thereof. The server(s) may also include database servers, including without limitation those commercially available from Oracle®, Microsoft®, Sybase®, and IBM® as well as open-source servers such as MySQL, Postgres, SQLite, MongoDB, and any other server capable of storing, retrieving, and accessing structured or unstructured data. Database servers may include table-based servers, document-based servers, unstructured servers, relational servers, non-relational servers, or combinations of these and/or other database servers.

The computing environment 100 can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen, or keypad) and at least one output device (e.g., a display device, printer, or speaker). Such a system may also include one or more storage devices, such as disk drives, optical storage devices, and solid-state storage devices such as random-access memory (“RAM”) or read-only memory (“ROM”), as well as removable media devices, memory cards, flash cards, etc.

Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims. While the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., may be either A or B or C, or any nonempty subset of the set of A and B and C. For instance, in the illustrative example of a set having three members, the conjunctive phrases “at least one of A, B, and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present. In addition, unless otherwise noted or contradicted by context, the term “plurality” indicates a state of being plural (e.g., “a plurality of items” indicates multiple items). The number of items in a plurality is at least two, but can be more when so indicated either explicitly or by context.

Operations of processes described herein can be performed in any suitable order unless otherwise indicated herein or otherwise clearly contradicted by context. Processes described herein (or variations and/or combinations thereof) may be performed under the control of one or more computer systems configured with computer-executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory. In some embodiments, the code is stored on set of one or more non-transitory computer-readable storage media having stored thereon computer-executable instructions that, when executed (i.e., as a result of being executed) by one or more processors of a computer system, cause the computer system to perform operations described herein. The set of non-transitory computer-readable storage media may comprise multiple non-transitory computer-readable storage media and one or more of individual non-transitory storage media of the multiple non-transitory computer-readable storage media may lack all of the code while the multiple non-transitory computer-readable storage media collectively store all of the code. Further, in some examples, the executable instructions are executed such that different instructions are executed by different processors. As an illustrative example, a non-transitory computer-readable storage medium may store instructions. A main CPU may execute some of the instructions and a graphics processor unit may execute other of the instructions. Generally, different components of a computer system may have separate processors and different processors may execute different subsets of the instructions.

Accordingly, in some examples, computer systems are configured to implement one or more services that singly or collectively perform operations of processes described herein. Such computer systems may, for instance, be configured with applicable hardware and/or software that enable the performance of the operations. Further, computer systems that implement various embodiments of the present disclosure may, in some examples, be single devices and, in other examples, be distributed computer systems comprising multiple devices that operate differently such that the distributed computer system performs the operations described herein and such that a single device may not perform all operations.

The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Embodiments of this disclosure are described herein, including the best mode known to the inventors for carrying out the invention. Variations of those embodiments may become apparent to those of ordinary skill in the art upon reading the foregoing description. The inventors expect skilled artisans to employ such variations as appropriate and the inventors intend for embodiments of the present disclosure to be practiced otherwise than as specifically described herein. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

It is noted that the terms “substantially” and “about” and “generally” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

The foregoing invention has been described in accordance with the relevant legal standards, thus the description is exemplary rather than limiting in nature. Variations and modifications to the disclosed embodiment may become apparent to those skilled in the art and fall within the scope of the invention.

1. A medical media and medical device ecommerce system substantially as described and illustrated.

2. A method of using a medical media and medical device ecommerce system substantially as described and illustrated.

3. A method comprising, by one or more computing devices:
uploading to the computing device value media by a contributor whereas the value media is media authored by a clinician and whereas the value media has intellectual value to other clinicians or individuals treated by clinicians;
assigning the value media one or more of a diagnosis and a procedure code;
validating the contributor's value media intellectual property (IP) ownership and referring contributors lacking IP ownership to a legal entity to procure protection;
creating an online value media store for the contributor containing a link to the value media;
designating a value media audience as either general public or medical professional;
designating a medical setting for the value media;
selecting a name for the value media; and
reviewing the value media for appropriate content.

4. The method by one or more computing devices of claim 3 whereby prior to the step of uploading of media from a contributor, the contributor inputs their professional license validation information.

5. The method by one or more computing devices of claim 3 further comprising:
validation of the value media by one or more professionals skilled in the respective medical discipline to assure the value media represents current accepted practices in the respective medical discipline.

6. The method by one or more computing devices of claim 5 further comprising:
performing an enhanced review of the value media by a professional skilled in the respective medical discipline wherein the professional integrates enhancements to the value media;
updating the intellectual property ownership; and
establishing a commission structure for creators of the value media.

7. The method by one or more computing devices of claim 6 further comprising:
validating one or both of the diagnosis and procedure code;
gathering value media and medical device statistical data; and
posting the statistical data to a statistical data report sales window.

8. The method by one or more computing devices of claim 5 further comprising:
posting of the value media to one or more of a value media professional database and general public use access database where it is made available for online purchase or complementary download in an ecommerce module.

9. The method by one or more computing devices of claim 8 further comprising:
a user searching through facing options one or more of the value media professional databases and general public databases based on at least one of: treatment type, treatment setting, keyword, diagnosis, and procedure codes;
making a purchase; and
calculating commissions and sending the commissions to the contributor.

10. The method by one or more computing devices of claim 8 further comprising:
choosing from facing options a device linked to the value media and adding it to a shopping cart of the ecommerce module.

11. The method by one or more computing devices of claim 3 further comprising:
entering a job board mode;
choosing from consumer facing options for a consumer seeking a medical professional comprising one or more filters such as, A) medical professional type, B) medical professional locational range, C) service center search, D) childcare requirements, E) advanced medical professional trainings, F) medical professional availability schedule, and G) consumer reviews of medical professionals.

12. The method by one or more computing devices of claim 3 further comprising:
entering a job board mode;
choosing from medical professional facing options for a medical professional seeking a consumer needing medical services comprising one or more filters such as, A) by medical practice discipline required, B) home care or service center location, C) by special patient needs, D) by medical professional qualifications, E) by area code or zip code location, and by keyword search.

13. The method by one or more computing devices of claim 3 further comprising:
entering a job board mode;
choosing from medical professional facing options a medical professional seeking peer review consortium opportunities search.

14. The method by one or more computing devices of claim 3 further comprising:
entering a job board mode;
choosing from medical professional facing options for a medical professional seeking a consumer search comprising one or more filters chosen from, A) medical professional type and locational range, B) medical professional advanced training, and C) medical professional availability schedule; and
reviewing by the consumer the medical professional provider options.

15. The method by one or more computing devices of claim 3 further comprising:
entering a job board mode;
utilizing a medical professional to consumer matcher and scheduler for in-home medical treatment, and service center patients;
viewing the match options; and
executing the match.

16. The method by one or more computing devices of claim 3 further comprising:
the medical professional treating the patient in the patient's home or at a medical service center.

17. The method by one or more computing devices of claim 16 further comprising providing childcare when the patient is treated at a medical service center.

18. The method by one or more computing devices of claim 3 further comprising:
entering a job board mode as a medical professional facing option; and
applying to become a medical service center.

19. The method by one or more computing devices of claim 3 further comprising:
entering a job board mode as a facing option; and
utilizing a Google Maps plugin as a filter to identify consumers or medical professionals, or consumers and medical professionals in a specific geographic location.

20. The method by one or more computing devices of claim 3 further comprising:
displaying a medical professional dashboard;
whereby said medical professional dashboard comprises display of one or more of the following, A) total count of value media uploaded, B) total count of value media that has been peer reviewed, C) value media uploaded listing and available for sale, D) value media for sale, compensation to date, cumulative sales, E) flagged value media that has been removed due to inappropriate content, F) value media uploaded but not yet published for sale, G) value media uploaded but not ready for sale, H) total CEUs (continuing education units) earned for peer reviewed and uploaded value media.

21. The method by one or more computing devices of claim 3 further comprising:
displaying a general public dashboard;
whereby said general public dashboard comprises display of one or more of the following with at least one display is greyed out, A) total count of value media uploaded, B) total count of value media that has been peer reviewed, C) value media uploaded listing and available for sale, D) value media for sale, compensation to date, cumulative sales, E) flagged value media that has been removed due to inappropriate content, F) value media uploaded but not yet published for sale, G) value media uploaded but not ready for sale, H) total CEUs (continuing education units) earned for peer reviewed and uploaded value media.

22. The method by one or more computing devices of claim 3 further comprising:
administering a CE (continuing education) test;
scoring the CE test;
pre-filling a CE certificate of completion; and
saving a copy of the CE certificate of completion for validation.
